(19) United States
(12) Patent Application Publication
(10) Pub. No.: US 2026/0114885 A1
(43) Pub. Date: Apr. 30, 2026

(54) SYSTEMS, DEVICES, AND METHODS FOR POSITIONING SHOCK WAVE EMITTERS CLOSER TO TARGET LESIONS IN THE BODY

(71) Applicant: Shockwave Medical, Inc., Santa Clara, CA (US)

(72) Inventors: Todd Weston JENKINS, San Jose, CA (US); Rainier BETELIA, San Jose, CA (US); Danielle Sheree ROND, Santa Clara, CA (US); Jason B. FRANCO, Milpitas, CA (US); Nancy P. NGUYEN, San Jose, CA (US); Alexandra VERNE, San Jose, CA (US); Jenna M. LIESKE, San Mateo, CA (US); David MOOSAVI, Castro Valley, CA (US); Lawrence John FORMOSA, Campbell, CA (US); Andrew James HUDON, Tabernash, CO (US); Thi Hong Nhung TRUONG, Lathrop, CA (US); Tim Ngoc HUYNH, Milpitas, CA (US); Ted Yun-Deh SU, Sunnyvale, CA (US)

(73) Assignee: Shockwave Medical, Inc., Santa Clara, CA (US)

(21) Appl. No.: 19/319,340

(22) Filed: Sep. 4, 2025

Related U.S. Application Data
(60) Provisional application No. 63/711,931, filed on Oct. 25, 2024.
(60) Provisional application No. 63/783,117, filed on Apr. 3, 2025.

(51) Int. Cl. A61B 17/22; A61B 17/00; A61B 90/00

(52) U.S. Cl. CPC A61B 17/22022; A61B 17/00234; A61B 90/39; A61B 2017/00238; A61B 2017/00305; A61B 2017/00539; A61B 2017/00557; A61B 2017/22062; A61B 2017/22065; A61B 2090/3966

(57) ABSTRACT

A catheter for generating shock waves includes a first elongate member; at least one shock wave emitter carried by the first elongate member; and an expandable member configured to move the at least one shock wave emitter laterally toward a lesion.

### CROSS REFERENCE TO RELATED APPLICATIONS

This application claims priority to U.S. Provisional Application No. 63/711,931, filed Oct. 25, 2024, and U.S. Provisional Application No. 63/783,117, filed Apr. 3, 2025, the contents of each of which are incorporated herein by reference in their entirety.

### FIELD OF THE DISCLOSURE

The present disclosure relates generally to the field of medical devices and methods, and more specifically to shock wave catheter devices for treating calcified lesions in body lumens and tissues, such as calcified lesions and occlusions in the cardiovascular system or on structural heart anatomy.

### BACKGROUND

The accumulation of calcium in a patient's blood vessels, tissues, or other organs can cause calcification that may disrupt organ function and lead to health issues for the patient. For example, when vascular plaque builds up along and in the walls of the coronary arteries, the accumulation can narrow the passageway of the vessel (referred to as stenosis) and restrict blood flow to the heart muscle, which can eventually lead to a heart attack. Treating stenosis is even more challenging when the plaque becomes hardened due to calcification.

A wide variety of catheters have been developed for treating stenotic blood vessels that are narrowed by the progressive growth and accumulation of plaque, a condition also known as atherosclerosis. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with a target lesion. The balloon is then pressurized (normally to greater than 10 atm), causing the balloon to expand in a vessel to push plaques back into the vessel wall and dilate occluded regions of vasculature. A particular focus is to treat calcified lesions of plaque in the vasculature associated with arterial disease. When treating calcified lesions, it is important to minimize damage to surrounding soft tissues while still breaking up the lesion as much as possible.

However, traditional dilation balloon angioplasty therapies may not work with calcified tissue because the calcium in the atherosclerotic plaque hardens the lesion, resisting the mechanical force of balloon expansion. The resistance can result in more procedural complication and vessel damage because the high-pressure balloons preferentially expand away from the hard calcified tissue. The predisposition of the ballon to expand in a direction of lower resistance increases the risk of major dissection or perforation of the vessel, often at the ends of a lesion at the interface between healthy tissue and calcified tissue (i.e., where the balloon encounters soft tissue). In the case of an eccentric calcified lesion where the hardened region is biased on a side of a vessel, the expansion ends up going preferentially in the direction opposite of the calcified region of the lesion, straining and dissecting the healthier side of the blood vessel. Moreover, in the case of nodular calcium, expansion of a standard angioplasty balloon can lead to pushing the node of calcified material in a manner that may puncture the vessel.

Another approach to dealing with calcified stenotic plaque is to cut away at a calcified lesion, by using a cutting or scoring balloon, an angioplasty balloon having a raised structure on the surface of the balloon (e.g., an angioplasty balloon with blade-like structures on its exterior). The expansion of an angioplasty balloon having a raised structure may allow for mechanical force on a lesion to be focused at the location of the raised structure, but these devices still do not provide for any protection from dissection or perforation resulting from preferential expansion of the balloon away from hardened tissue. Another technique for cutting away at a calcified lesion is by using an atherectomy device, which typically includes a motor-driven rotating or oscillating blade that is pushed into and cuts through an occlusion (also referred to as “debulking” or “extirpation”). Because these treatments work by liberating the calcified tissues from a blood vessel wall, there is an increased risk of embolism where the free-floating masses of calcification may proceed down the blood stream. Such systems may include baskets to capture or negative-pressure lumens to aspirate such unmoored emboli as a necessary additional structure to ensure the safety of such devices. An additional concern for atherectomy devices is that the movement or rotation of atherectomy catheter blades generates frictional heat and can cause a related thermal injury from mere operation of the atherectomy device. That heat can directly injure the lining of a blood vessel and can also lead to an increased risk of blood clotting. Naturally, the action of a moving blade within the vasculature also significantly increases the potential for a large dissection and perforation of the blood vessel by the blade itself.

Accordingly, there is an ongoing need for improved medical devices and treatments to address calcification and restore organ function. One such treatment is intravascular lithotripsy (IVL), which uses acoustic pressure to break up the calcified regions. In IVL, a device such as a catheter is advanced within the patient's body to a position adjacent to the treatment area. The IVL device is configured to generate acoustic waves, specifically, ultrasonic short pulse waves (also known as “shock waves”), which propagate outward from the IVL device to modify the calcified regions. The acoustic pressure of the shock waves may crack and disrupt the calcified regions near the IVL device without harming the surrounding blood vessels, tissues, or other organs. In particular, IVL can address and treat calcified plaques and stenosis with a safety profile that minimizes risk of blood vessel damage and with an efficacy profile that provides for durable circulatory restoration

Conventional shock wave catheters may be less effective for treating lesions in large blood vessels and valves. Catheters should be sufficiently small to maneuver through vessels that the catheters traverse to reach the large blood vessels and valves. Once in the large blood vessels and valves, a balloon of the catheter is inflated to a relatively large diameter that moves the walls of the balloon farther from the emitters within the balloon, which are often located along a central tube running along the longitudinal axis of the catheter. Accordingly, when shock waves are emitted, they may need to propagate farther in larger blood vessels to reach lesions than in smaller blood vessels, which may reduce the force applied to crack the lesions. To address the relatively lower force of the shock waves as they propagate further from the emitters inside large balloons, some systems increase the supplied power to the emitters to produce higher magnitude shock waves in the balloon. However, increasing the magnitude of the shock waves requires increased power and may introduce other issues, such as increased wear on the emitters of the device and a risk of rupturing the balloon wall with high voltage shock waves. Other treatment protocols for large vessels and valves may require longer shock wave treatments, which risks ischemia and other complications during an angioplasty procedure.

### SUMMARY

Disclosed herein are devices, systems, and methods for positioning shock wave emitters relatively closer to target lesions within the body than conventional shock wave catheters. The catheters described herein include an expandable member configured to push an elongate member carrying at least one shock wave emitter closer to a target lesion when expanded. Radiopaque markers may be disposed on the catheter and arranged such that a user can observe the orientation of the shock wave emitter(s) and reorient the catheter as needed to position the shock wave emitter(s) closer to the target lesion.

According to an aspect, an exemplary catheter for generating shock waves comprises: a first elongate member; an enclosure mounted to the first elongate member; at least one shock wave emitter carried by the first elongate member and disposed within the enclosure; a first set of radiopaque markers positioned on the first elongate member within the first enclosure; a second elongate member; an expandable member mounted to the second elongate member and configured to expand to contact the enclosure mounted to the first elongate member; and a second set of radiopaque markers positioned on the second elongate member within the expandable member, wherein the second set of radiopaque marks has a different arrangement than the first set of radiopaque markers.

Optionally, the first elongate member extends distally from a catheter body. Optionally, the first elongate member is a distal portion of a catheter body. Optionally, the different arrangement of radiopaque markers comprises a different number of radiopaque markers. Optionally, the different arrangement of radiopaque markers comprises a different spacing between the radiopaque markers. Optionally, the first set of radiopaque markers and the second set of radiopaque markers are arranged on the first and second elongate members such that: the first set of radiopaque markers appears colinear with the second set of radiopaque markers when viewed from a first perspective using radiographic imaging. Optionally, the first set of radiopaque markers appears non-colinear with the second set of radiopaque markers when viewed from a second perspective using radiographic imaging. Optionally, the enclosure is non-compliant. Optionally, the expandable member mounted to the second elongate member is an enclosure configured to expand when inflated with a fluid. Optionally, the catheter comprises a first fluid conduit for filling the enclosure mounted to the first elongate member and a second fluid conduit for filling the enclosure mounted to the second elongate member so that the enclosure mounted to the first elongate member can be filled independently of the enclosure mounted to the second elongate member. Optionally, the first elongate member comprises a guidewire lumen. Optionally, the second elongate member comprises a guidewire lumen. Optionally, either or both of the first elongate member and the second elongate member comprises a guidewire lumen. Optionally, a distal end of the first elongate member is free of a distal end of the second elongate member. Optionally, the first elongate member and the second elongate member extend proximally within the same lumen along at least a portion of the length of the catheter body. Optionally, the at least one shock wave emitter is located on an opposite side of the first elongate member from the second elongate member. Optionally, the expandable member mounted to the second elongate member is an expandable frame configured to allow a fluid to pass through the frame when the frame is expanded. Optionally, the catheter comprises a moveable shaft configured such that translation of the moveable shaft in a first direction causes the expandable frame to expand and translation of the moveable shaft in a second direction causes the expandable frame to collapse. Optionally, the expandable frame comprises at least one of a wire frame wire and a mesh. Optionally, the expandable frame is configured to be self-expanding. Optionally, a distal end of the expandable member is positioned proximally of a distal end of the enclosure.

According to an aspect, an exemplary method for positioning a shock wave generating catheter closer to a target lesion comprises: advancing a catheter within a lumen to the target lesion; orienting the catheter within the lumen such that a first elongate member comprising a first set of radiopaque markers is positioned relatively closer to the target lesion than a second elongate member comprising a second set of radiopaque markers; moving a first elongate member of the catheter closer to the target lesion by expanding an expandable member such that the expandable member pushes the first elongate member closer to the target lesion; and generating one or more shock waves using at least one shock wave emitter of the catheter positioned on the first elongate member.

Optionally, the method includes rotating the catheter to position the first elongate member of the catheter adjacent to the target lesion. Optionally, the expandable member comprises an enclosure, and wherein expanding the expandable member connected to the second elongate member comprises inflating the enclosure mounted to a distal end of the second elongate member to contact an enclosure mounted to the first elongate member. Optionally, the expandable member comprises an expandable frame, and wherein expanding the expandable member comprises moving a movable shaft connected to the expandable frame in a proximal direction. Optionally, the at least one shock wave emitter of the catheter is positioned within an enclosure connected to the first elongate member.

A catheter for generating shock waves comprises: a catheter body; a tube-shaped enclosure sealed to a distal end of the catheter body, the tube-shaped enclosure comprising an outer cylindrical wall, an inner cylindrical wall, and a fillable region between the outer cylindrical wall and the inner cylindrical wall configured to be filled with a fluid, wherein a distal portion of the catheter body is positioned within the fillable region; and at least one shock wave emitter disposed on the catheter body within the fillable region between the outer cylindrical wall and the inner cylindrical wall.

Optionally, the inner cylindrical wall defines an open channel when the tube-shaped enclosure is filled with a fluid. Optionally, the open channel is configured to allow body fluid flowing within a body lumen to flow through the open channel when the catheter is disposed in the lumen and the tube-shaped enclosure is filled with a fluid. Optionally, the tube-shaped enclosure is semi-compliant. Optionally, the tube-shaped enclosure is non-compliant balloon. Optionally, a compliance of the outer cylindrical wall is different than a compliance of the inner cylindrical wall. Optionally, a distal portion of the tube-shaped enclosure comprises a tapered region. Optionally, the outer cylindrical wall has a diameter of between 10 millimeters and 30 millimeters. Optionally, the inner cylindrical wall has a diameter of at least 6 millimeters. Optionally, the tube-shaped enclosure comprises a proximally extending cylindrical wall at a proximal end of the balloon and a distally extending cylindrical wall at a distal end of the balloon, wherein the proximally extending cylindrical wall and the distally extending cylindrical wall are sealed to the catheter body. Optionally, the tube-shaped enclosure comprises a shape memory material. Optionally, catheter comprises a braided outer shaft on a proximal portion of the catheter body. Optionally, the at least one shock wave emitter is configured to emit shock waves toward the outer cylindrical wall. Optionally, the at least one shock wave emitter is positioned at circumferential location of the catheter body that is closest to the outer cylindrical wall.

According to an aspect, an exemplary method for positioning a shock wave generating catheter closer to a target treatment area, comprises: advancing a catheter comprising a tube-shaped enclosure mounted to a catheter body within a lumen; positioning a distal portion of the catheter such that at least one shock wave emitter of the catheter is positioned adjacent to a target treatment area; filling a tube-shaped enclosure to form an open channel through which body fluid flows; generating one or more shock waves using at least one shock wave emitter of the catheter.

Optionally, the method includes: deflating the tube-shaped enclosure; rotating the catheter to position the at least one shock wave emitter adjacent to a different target treatment area; filling the tube-shaped enclosure; and generating one or more additional shock waves using the at least one shock wave emitter of the catheter. Optionally, positioning a distal portion of the catheter such that at least one shock wave emitter of the catheter is positioned adjacent to a target treatment area comprises: positioning the distal portion of the catheter across a valve annulus. Optionally, positioning a distal portion of the catheter such that at least one shock wave emitter of the catheter is positioned adjacent to a target treatment area comprises: positioning the distal portion of the catheter adjacent to an aortic valve leaflet or a mitral valve leaflet.

According to an aspect, an exemplary catheter for generating shock waves comprises: an elongate member; an enclosure mounted to the first elongate member; at least one shock wave emitter carried by the first elongate member and disposed within the enclosure; a first expandable member extending away from the elongate member in a first direction and configured to expand to contact the enclosure mounted to the elongate member; and a second expandable member extending away from the elongate member and the first expandable member in a second direction and configured to expand to contact the enclosure mounted to the elongate member.

Optionally, the first expandable member is mounted to a second elongate member and the second expandable member is mounted to a third elongate member. Optionally, the first expandable member and the second expandable member have a substantially equal outer diameter when filled with a fluid. Optionally, the enclosure has substantially the same outer diameter as the first expandable member and the second expandable member when filled with a fluid. Optionally, at least one radiopaque marker is positioned on the elongate member. Optionally, a distal end of the first expandable member, a distal end of the second expandable member, and a distal end of the enclosure are connected.

According to an aspect, an exemplary system for generating shock waves comprises: a shock wave energy generator; and the catheter of any of the examples described herein.

According to some aspects, a catheter for generating shock waves comprises: a first elongate member; an enclosure mounted to the first elongate member; at least one shock wave emitter carried by the first elongate member and disposed within the enclosure; a second elongate member; and an expandable frame mounted to the second elongate member and configured to expand to contact the enclosure mounted to the first elongate member, wherein the expandable frame is configured such that, when the catheter is located in a blood vessel and the expandable frame is expanded, blood can flow through the expandable frame. Optionally, the expandable frame comprises a plurality of expandable members. Optionally, the expandable frame comprises a coiled member, a wire frame, or a mesh. Optionally, the catheter includes a moveable shaft connected to the expandable frame configured to expand and collapse the expandable frame.

According to some aspects, a catheter for generating shock waves comprises: an inner elongate member; an outer elongate member extending radially outwardly of the inner elongate member, wherein the outer elongate member is translatable relative to the inner elongate member between a retracted position and an extended position; at least one support member positioned at least partially between the inner elongate member and the outer elongate member when the outer elongate member is in the extended position, wherein the at least one support member is configured to move outwardly from the inner elongate member when the outer elongate member is translated from the extended position to the retracted position; and at least one shock wave emitter mounted to the at least one expandable emitter support member.

Optionally, at least one support member comprises a pre-shaped memory material configured to move outwardly from the inner elongate member when the outer elongate member is translated from the extended position to the retracted position. Optionally, the at least one support member comprises a lumen, wherein the pre-shaped memory material is positioned within the lumen. Optionally, the catheter includes a user-engageable locking member configured to lock the outer elongate member to the inner elongate member such that the outer elongate member is not translatable relative to the inner elongate member. Optionally, rotating the locking member in a first direction locks the outer elongate member to the inner elongate member. Optionally, the at least one support member is positioned within at least one groove of the inner elongate member. Optionally, the catheter includes an enclosure mounted to the at least one expandable member, wherein the at least one shock wave emitter is positioned within the enclosure.

According to some aspects, a method for treating lesions within the body comprises: advancing a catheter within a body lumen to a target lesion; sliding an elongate member proximally to deploy one or more elongate support members such that the one or more elongate support members move outwardly from a longitudinal axis of the catheter; introducing a conductive fluid into one or more enclosures provided on the one or more elongate support members; generating one or more shock waves using at least one shock wave emitter positioned on at least one of the one or more elongate support members.

Optionally, the method includes rotating a locking member to unlock the outer elongate member from a second elongate member prior to sliding the outer elongate member proximally. Optionally, the method includes manipulating the catheter to position a portion of each of the one or more elongate support members in a respective valve cusp. Optionally, the method includes sliding the elongate member distally to collapse the elongate support members inwardly. Optionally, the fluid is introduced into each of the one or more enclosures independently of one or more of the other enclosures. Optionally, he fluid is introduced into each of the one or more enclosures simultaneously.

In some implementations, shock wave treatment of heart valve anatomy can be used as a preparatory procedure, to optimize the tissue region for receipt and implantation of a replacement heart valve.

According to some aspects, a catheter for generating shock waves comprises: an inner elongate member; an outer elongate member extending radially outwardly of the inner elongate member, wherein the inner elongate member is translatable relative to the outer elongate member between a retracted position and an extended position; at least one support member positioned at least partially between the inner elongate member and the outer elongate member when the inner elongate member is in the retracted position, wherein the at least one support member is configured to move outwardly from the inner elongate member when the inner elongate member is translated from the retracted position to the extended position; and at least one shock wave emitter mounted to the at least one expandable emitter support member.

Optionally, at least one support member comprises a pre-shaped memory material configured to move outwardly from the inner elongate member when the outer elongate member is translated from the extended position to the retracted position. Optionally, the at least one support member comprises a lumen, wherein the pre-shaped memory material is positioned within the lumen. Optionally, the catheter comprises a user-engageable locking member configured to lock the outer elongate member to the inner elongate member such that the inner elongate member is not translatable relative to the outer elongate member. Optionally, rotating the locking member in a first direction locks the outer elongate member to the inner elongate member. Optionally, rotating the locking member in a second direction unlocks the outer elongate member from the inner elongate member. Optionally, the at least one support member is positioned within at least one groove of the inner elongate member. Optionally, the catheter comprises an enclosure mounted to the at least one expandable member, wherein the at least one shock wave emitter is positioned within the enclosure.

In some embodiments, any one or more of the characteristics of any one or more of the systems and methods recited above may be combined, in whole or in part, with one another and/or with any other features or characteristics described elsewhere herein.

### DESCRIPTION OF THE FIGURES

The invention will now be described, by way of example only, with reference to the accompanying drawings, in which:

FIG. 1 illustrates an exemplary system for generating shock waves according to some examples.

FIG. 2A illustrates an exemplary catheter with shock wave emitters and an expandable member viewed from a first perspective according to some examples.

FIG. 2B illustrates the exemplary catheter of FIG. 2A viewed from a second perspective according to some examples.

FIG. 3A illustrates an exemplary shock wave catheter with shock wave emitters and an expandable member in a collapsed configuration within a valve according to some examples.

FIG. 3B illustrates the exemplary shock wave catheter of FIG. 3A with the expandable member in an expanded configuration according to some examples.

FIG. 4A illustrates an exemplary shock wave catheter with shock wave emitters and an expandable frame in a collapsed configuration according to some examples.

FIG. 4B illustrates the exemplary shock wave catheter of FIG. 4A with the expandable frame in an expanded configuration according to some examples.

FIG. 5 illustrates an exemplary method for positioning shock wave emitters closer to a lesion and generating shock waves according to some examples.

FIG. 6A illustrates an isometric view of an exemplary catheter with shock wave emitters and a tube-shaped enclosure according to some examples.

FIG. 6B illustrates a view facing the distal end of the catheter of FIG. 6A according to some examples.

FIG. 7 illustrates aspects of a catheter positioned inside a tube-shaped enclosure according to some examples.

FIG. 8 illustrates aspects of a catheter positioned inside another tube-shaped enclosure according to some examples.

FIG. 9 illustrates another exemplary method for positioning shock wave emitters closer to a lesion and generating shock waves according to some examples.

FIGS. 10A-10B illustrate aspects of another with shock wave emitters and expandable members according to some examples.

FIG. 11 illustrates an exemplary computing system according to some examples.

FIG. 12 illustrates aspects of an exemplary catheter that includes an exemplary expandable frame according to some examples.

FIG. 13 illustrates aspects of an exemplary catheter that includes an exemplary expandable frame according to some examples.

FIGS. 14A-14D illustrate aspects of an exemplary catheter that includes laterally moveable shock wave emitter support members that can be used to position shock wave emitters closer to lesions, according to some examples.

FIG. 15 illustrates aspects of an aortic valve according to some examples.

FIG. 16 illustrates a flowchart representing steps of a method for generating shock waves using one or more of the catheters disclosed herein according to some examples.

FIG. 17 illustrates aspects of an exemplary method for positioning shock wave emitters relatively closer to a lesion according to some examples.

FIG. 18 illustrates aspects of an exemplary catheter including a plurality of expandable members connected at a distal end according to some examples.

FIG. 19 illustrates aspects of an exemplary method for positioning shock wave emitters relatively closer to a lesion according to some examples.

FIGS. 20A-20B illustrates aspects of an exemplary catheter including an expandable frame positioned radially outward of a plurality of shock wave emitters according to some examples.

FIG. 21 illustrates aspects of an exemplary method for treating a lesion using shock waves according to some examples.

FIG. 22 illustrates aspects of an exemplary catheter including an expandable frame and an expandable enclosure for moving shock wave emitters closer to a lesion according to some examples.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

In recent years, in order to treat atherosclerosis and related conditions, the technique and treatment of intravascular lithotripsy (“IVL”) has been developed, which is an interventional procedure to modify calcified plaque in diseased vasculature. More precisely, IVL is the energy-based generation of ultrasonic acoustic pressure waves for modification, fracture, and fragmentation of vascular calcification in situ. The mechanism of plaque modification is through use of a catheter having one or more ultrasonic short pressure pulses (commonly referred to as “shock waves”) emit from a generating source located within a liquid that can create acoustic ultrasonic shock waves that modify and fracture the calcified plaque. IVL improves arterial compliance and enables optimal lumen expansion in vascular interventional procedures. IVL devices vary in design with respect to the energy source used to generate the acoustic shock waves, with two exemplary energy sources being electrohydraulic generation and laser generation. Moreover, the broader application of intracorporeal lithotripsy, leveraging the systems and techniques of IVL, can be used for treatment of other tissues and organs within a patient's body, as in the present disclosure for the treatment of structural heart anatomy.

For electrohydraulic generation of ultrasonic short pressure pulses, a conductive solution (e.g., saline) can be contained within an enclosure that surrounds electrodes or can be flushed through a tube that surrounds the electrodes. The calcified plaque modification is achieved by creating ultrasonic shock waves within the catheter by an electrical discharge (e.g., a plasma arc) across the electrodes. The energy from this electrical discharge enters the surrounding fluid, generating an acoustic shock wave where the wave itself is ultrasonic (i.e., a wave that has frequency components of greater than 20,000 Hz). In addition, the discharge creates one or more rapidly expanding and collapsing vapor bubbles that generate secondary shock waves due to the cavitation of the collapsing vapor bubble. The shock waves propagate radially outward and modify calcified plaque within the blood vessels. The shock waves travel deeply and safely through soft arterial tissue because of the acoustic impedance soft tissue, which is similar to water. Acoustic impedance is a function of the density and the elasticity of a material and the speed of sound through that material. When the shock waves encounter tissues with a different acoustic impedance, such as intimal calcification of plaque close to the surface or endothelium of a vessel or medical calcification in the smooth muscle layer of a vessel, the leading edge of the shock wave imparts compressive stress on the calcified tissue. Shearing occurs on the lesion as the shock wave passes through the calcification. When the shock wave reaches the distal boundary of the calcification, the shock wave is both transmitted and reflected, inducing tensile stress that pulls the calcification apart. Further compressive stress is applied by the squeezing which occurs when the ultrasonic shock wave entering the calcium propagates faster than the remaining shock wave travelling outside the calcified region of tissue. These forces generated by IVL result in multi-plane and longitudinal fractures of the calcification in the tissue.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with conductive fluid (e.g., using a relatively low pressure of 2-4 atm) so that the balloon expands to contact the lesion but not to a degree that substantively displaces the lesion. Voltage pulses can then be applied across the electrodes of electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can include electrodes disposed within a closed volume other than an angioplasty balloon, such as a cap, balloons of variable compliancy, or other type of enclosure.

Critically, the calcified plaque remains in place following the shock waves; for IVL intimal calcium remains in the blood vessel lining and medical calcium remains in the muscle tissue surrounding the blood vessel. IVL generally does not cause the debulking or extirpation of tissue from a blood vessel wall. Similarly, for calcification of structural heart tissues such as organ walls, arteries and veins, valve leaflets, commissures, and the like, calcification that is fragmented by ultrasonic short pressure pulses does not separate from the surface of the lesion, but remains within the target tissue while the target tissue has become more pliable and flexible.

Accordingly, the IVL process can also be considered different from standard atherectomy procedures and different from cutting or scoring balloons at least in that IVL cracks calcium but does not liberate the calcium from the tissue. Hence, generally speaking, IVL systems should not require aspiration nor embolic protection. Accordingly, IVL does not carry the same degree of risk of embolism, perforation, dissection, or other damage to vasculature as atherectomy procedures or angioplasty procedures using cutting or scoring balloons. In further contrast with cutting techniques, due to the compliance of a normal blood vessel and non-calcified plaque, the shock waves produced by IVL do not modify the normal healthy vessel tissue or non-calcified plaque. In other words, the shock waves from IVL do not have an adverse clinical impact on soft tissues while treating the hardened calcified anatomy.

For laser generation of acoustic shock waves, a laser pulse is transmitted into and energy from the laser is absorbed by a fluid within the catheter, optionally with a target to act as catalyst for the laser absorption. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of electrohydraulic and laser-based IVL devices are not intended to be a comprehensive list of potential energy sources to create the ultrasonic IVL shock waves.

Disclosed herein are devices, systems, and methods for positioning shock wave emitters near target lesions within the body. An exemplary device includes an expandable member configured to push at least one shock wave emitter closer to a target lesion. The device may also include radiopaque markers configured to enable a user to determine the position of the shock wave emitters relative to the target lesion and to reorient the catheter as needed to reposition the shock wave emitters closer to the lesion within the body.

In some examples, a catheter includes a first elongate member and a second elongate member. An enclosure may be mounted to the first elongate member, and at least shock wave emitter may be carried by the first elongate member and disposed within the enclosure. An expandable member may be mounted to the second elongate member and configured to expand to contact the enclosure mounted to the first elongate member. The expandable member can push the first elongate member, and thus the shock wave emitter(s) on the first elongate member, laterally toward the target lesion.

Radiopaque markers may be included on the first and second elongate members and configured to enable a user to distinguish between the two elongate members and determine the relative positioning of the two elongate members within the lumen. The radiopaque markers provide a user with visual indications of where the elongate member carrying the shock wave emitter(s) is within a lumen based on the relative positioning of the two elongate members indicated by the radiopaque markers. A first set of radiopaque markers may be positioned on the first elongate member within the first enclosure and a second set of radiopaque markers may be positioned on the second elongate member within (e.g., radially inward of) the expandable member. The second set of radiopaque marks may have a different arrangement (e.g., different number, different spacing, etc.) than the first set of radiopaque markers. When viewed from a first perspective using radiographic imaging, the first set of radiopaque markers appears colinear with the second set of radiopaque markers. When viewed from a second perspective using radiographic imaging, the first set of radiopaque markers appears non-colinear with the second set of radiopaque markers. The user can rotate the catheter to reorient the shock wave emitter(s) closer to a target lesion based on the position of the two sets of radiopaque markers. Once reoriented closer to the target lesion, the shock wave emitter(s) can be pushed laterally outward toward the lesion using the expandable member.

In some examples, a catheter includes a tube-shaped enclosure sealed to a distal end of an elongate member of the catheter such that a distal portion of the elongate member carrying at least one shock wave emitter is positioned within a fillable region of the tube-shaped enclosure, between inner and outer walls of the enclosure. When the tube-shaped enclosure is filled, the elongate member carrying the at least one shock wave emitter may be pushed laterally toward a target lesion within a body lumen. An inner wall of the tube-shaped enclosure defines an open channel that allows fluid flowing within the body lumen to flow past the catheter while the tube-shaped enclosure is filled. Shock waves can thus be generated relatively more closely to the lesion while still permitting flow through the body lumen.

As a form of therapy, shock wave treatment of heart valve anatomy can be used as a preparatory procedure, to optimize the tissue region for receipt and implantation of a replacement heart valve. More specifically, heart valve implants, both mechanical and tissue-based, can be challenging to implant and seat on the target location where the target tissue is hardened due to calcification. The application of ultrasonic short pressure pulses prior to a replacement valve implantation procedure can make the target location more pliable and flexible, thereby allowing an implant to more easily access and orient at the implant location. Moreover, the relatively pliable and flexible heart valve tissue can allow for the implant to securely sit or anchor at and around the annular region of the valve, thereby forming a better seal at the perimeter of the valve implant, reducing any seepage of leakage of fluid going around the main passage of the valve implant. Accordingly, the application of intracorporeal lithotripsy to a heart valve can provide for improved performance and longevity of a valve implant.

Notably, ultrasonic short pressure pulses have a non-ablative mechanism of action, unlike other structural heart therapies such as radiofrequency ablation (heat-based) or cryoablation. Of course, ultrasonic short pressure pulses delivered to structural heart tissues can be used in combination with ablation-based therapies as appropriate.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an electrode assembly. In the context of the present disclosure, the term “emitter” broadly refers to the region of an electrode assembly where the current transmits across the electrode pair, generating a shock wave. The terms “emitter sheath” and “emitter band” refers to a continuous or discontinuous band of conductive material that may form one or more electrodes of one or more electrode pairs, thereby forming a location of one or more emitters.

Components of emitters, including electrodes and emitter sheaths/bands, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

For treatment of an occlusion in a blood vessel, the voltage pulse applied by a power source, including any of the power sources described herein (which may also be referred to herein as voltage sources or pulse generators), is typically in the range of from about five hundred to three thousand volts (500 V-3,000 V). In some implementations, for the treatment of stenosis in a blood vessel or of another anatomical feature (e.g., structural heart tissues), the voltage pulse applied by the voltage source can be up to about ten thousand volts (10,000 V), up to about fifteen thousand volts (15,000 V), or higher than fifteen thousand volts (15,000 V). The pulse width of the applied voltage pulses ranges between one microsecond and six microseconds (1-6 μs). The repetition rate or frequency of the applied voltage pulses may be between about 1 Hz and 10 Hz. The total number of pulses applied by the power source may be, for example, sixty (60) pulses, eighty (80) pulses, one hundred twenty (120) pulses, three hundred (300) pulses, or up to five hundred (500) pulses, or any increments of pulses within this range.

Alternatively, or additionally, in some examples, the power source may be configured to deliver a packet of micro-pulses (e.g., 500 micro-pulses in a packet) having a sub-frequency between about 10 Hz-10 kHz. The preferred voltage, repetition rate, and number of pulses may vary depending on, e.g., the size of the lesion, the extent of calcification, the size of the blood vessel, the attributes of the patient, or the stage of treatment. For instance, a physician may start with low energy shock waves and increase the energy as needed during the procedure, or vice versa. The amount of power delivered for shock waves may further vary during the course of a procedure, following a predetermined sequence of energy increases or decreases, or by changing the amount of energy delivered in response to sensor data obtained prior to and/or during the IVL treatment procedure. The magnitude of the shock waves can be controlled by controlling the voltage, current, duration, and repetition rate of the pulsed voltage from the power source.

In some implementations, an IVL catheter may be a “rapid exchange-type” (“RX”) catheter provided with an opening portion through which a guidewire can be guided (such as through a middle portion of a central tube in a longitudinal direction). In some other implementations, an IVL catheter may be an “over-the-wire-type” (“OTW”) catheter in which a guidewire lumen is formed throughout the overall length of the catheter, and a guidewire can be guided through the proximal end of a hub. A guidewire lumen entry point to a catheter is at or proximate to the distal end of the catheter tip, and the guidewire lumen extends through a portion of the catheter to an exit port. Thus in use, a guidewire is delivered into the anatomy of a patient, the proximal end of the guidewire (outside the patient) is fed into the distal end opening of the catheter, and the catheter is run along the guidewire until it reaches the target tissue at the distal end of the guidewire (inside the patient); the effective difference between an OTW and an Rx catheter is where the guidewire exits the catheter. The selection between an OTW design and an Rx design is driven by factors including (but not limited to): anatomy to be treated (e.g., coronary vasculature vs. peripheral vasculature); the length of guidewire to be used; the trackability, stiffness, torque transmission, and deliverability of the catheter; the profile and cross-section of the catheter, the ability to exchange a wire when the catheter is past a stenosis; positioning of the distal end of a catheter close to the end of a guidewire and further obtaining positional confirmation of the catheter.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

Although shock wave devices described herein generate shock waves based on high voltage applied to electrodes, it should be understood that a shock wave device additionally or alternatively may comprise a laser and optical fibers as a shock wave emitter system whereby the laser source delivers energy through an optical fiber and into a fluid to form shock waves and/or cavitation bubbles.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradation within the ranges set forth relative to the given dimension or measurement. It should be further appreciated that any disclosure of a numerical range as a boundary term or inequality term is similarly inclusive of any numerical increment or gradation within the given range; e.g., recitation of a parameter that is “at least a defined value, where the defined value ranges from 5% to 50%” supports the disclosure of that parameter being “at least 5%”, “at least 50%”, “at least 37%”, “at least 42.4%”, and the like. Furthermore, numerical designators such as “first”, “second”, “third”, “fourth”, etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

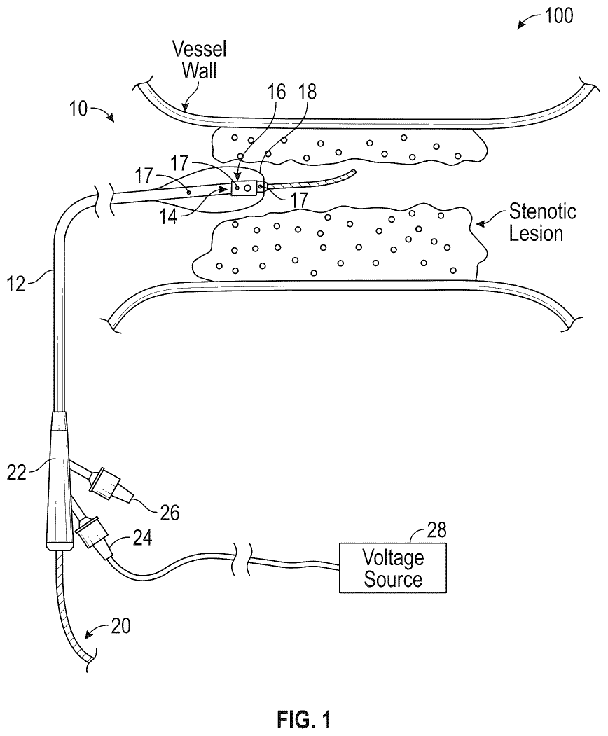

FIG. 1 illustrates a system 100 for treating calcifications in body lumens. The system includes a shock wave generating catheter 10. The catheter 10 may be used to fragment, crack, or otherwise break up calculi within a body lumen, for instance, to treat various occlusions within blood vessels. The catheter 10 includes a plurality of shock wave emitters 16 positioned within an enclosure 18. The catheter 10 is advanced into an occlusion in a patient's vasculature, such as the stenotic lesion depicted in FIG. 1, over a guidewire 20 carried in a guidewire sheath and voltage pulses are applied to the shock wave emitters 16 to generate shock waves. The shock wave emitters 16 each include electrode pairs having first and second electrodes separated by a gap at which shock waves are formed when a current flows across the gap between the electrodes (i.e., when a voltage is applied across the first and second electrodes).

An enclosure 18 is sealably attached to the distal end 14 of the catheter 10, forming an annular channel around a body 12 of the catheter 10. The enclosure 18 surrounds the plurality of shock wave emitters 16. The enclosure 18 can be filled or inflated with a conductive fluid, such as saline. The enclosure 18 can be compliant (e.g., a low-profile flexible angioplasty balloon, a polymer membrane in tension that can flex outward, etc.) such that it expands when filled or may be noncompliant such that it maintains a substantially constant volume and profile when filled. The conductive fluid enables current to flow across the electrodes of the shock wave emitters to generate shock waves. The shock waves propagate within the conductive fluid outwardly from the electrode pairs of the shock wave emitters 16 through the walls of the enclosure 18 and then into the target lesion. In one or more examples, the conductive fluid may also contain x-ray contrast fluid for fluoroscopic viewing of the catheter 10 during use. The enclosure 18 may mitigate thermal injury to soft tissue and reduce cavitation stresses by limiting expansion of the vapor bubbles produced during shock wave generation. For instance, the vapor bubbles hit the enclosure wall before reaching their maximum potential size, thus inducing collapse, and reducing cavitation stress and preventing soft tissue injury that can be caused by tensile stresses during cavitation bubble collapse.

The catheter 10 includes a proximal end 22 (which may include or form a handle) that remains outside of a patient's vasculature during treatment. The proximal end 22 may include an entry port for receiving the guidewire 20. The proximal end 22 may include at least one fluid port 26, which may be used for filling and emptying the enclosure 18 during treatment. An electrical connection port 24 is also located on the proximal end 22 to provide an electrical connection between the distal shock wave emitters 16 and an external voltage source 28.

The catheter body 12 extends from the proximal end 22 to the distal end 14 of the catheter. The catheter body 12 provides various internal conduits connecting elements of the distal end 14 with the proximal end 22 of the catheter. The catheter body 12 includes an elongate tube that includes a lumen for receiving the guidewire 20. The elongate tube may include additional lumens extending through the catheter body 12 or along an outer surface of the catheter body 12. For example, one for fluid lumens (e.g., a fluid inlet lumen and a fluid outlet lumen or a combined flush lumen) can be located along or within the catheter body 12 for carrying conductive fluid from the fluid port 26 into the enclosure 18.

In some examples, one or more sensors 17 are positioned along the catheter 10. The sensors 17 may be positioned at any location on catheter 10. For instance, the sensors 17 may be positioned proximal to one or more shock wave emitters 16, distal from one or more shock wave emitters 16, and/or intermediary between one or more shock wave emitters 16 (or any combination thereof). The sensors 17 may be positioned external to the enclosure 18 and/or outside of a patient. For instance, certain sensors, such as a pressure sensor, may be positioned outside of the enclosure 18 and/or the patient to measure pressure on the system as a whole when components are in fluid communication. The sensors may include one or more of any suitable sensor devices, such as a pressure sensor, a thermal sensor, an electrical sensor (e.g., current, voltage, resistance, and/or impedance sensors), or a visualization element. Sensors 17 can provide feedback to an operator using catheter 10 by measuring parameters in the surrounding environment and thereby indicating a status of the catheter 10 and components thereof, and further providing for guidance on what further steps the operator may decide to implement with catheter 10. For example, in implementations where sensors 17 include pressure sensors, a slight decrease in pressure may indicate success at cracking a calcified lesion, due to the fact that the expandable member surrounding the emitters is able to further expand without changing the volume of fluid within the expandable member. Further, a significant decrease in pressure may indicate a rupture failure mode where the expandable member has lost seal and fluid volume, and thus guiding toward withdrawal of the device. In implementations where the sensor devices include a visualization element, an operator of the catheter 10 may be able to more clearly understand where the catheter 10 is located relative to a target lesion or anatomy, prior to, during, and after delivering therapy.

In some implementations, the material that forms the primary surface(s) of the enclosure 18 through which shock waves pass can be a noncompliant polymer. In other implementations, a rigid and inflexible structure may be used in lieu of enclosure 18. The enclosure 18 may mitigate thermal injury to soft tissue and reduce cavitation stresses by limiting expansion of the vapor bubbles produced during shock wave generation to the interior of the enclosure. For instance, the vapor bubbles hit the enclosure wall before reaching their maximum potential size, thus inducing collapse, and reducing cavitation stress and preventing soft tissue injury that can be caused by tensile stresses during cavitation bubble collapse.

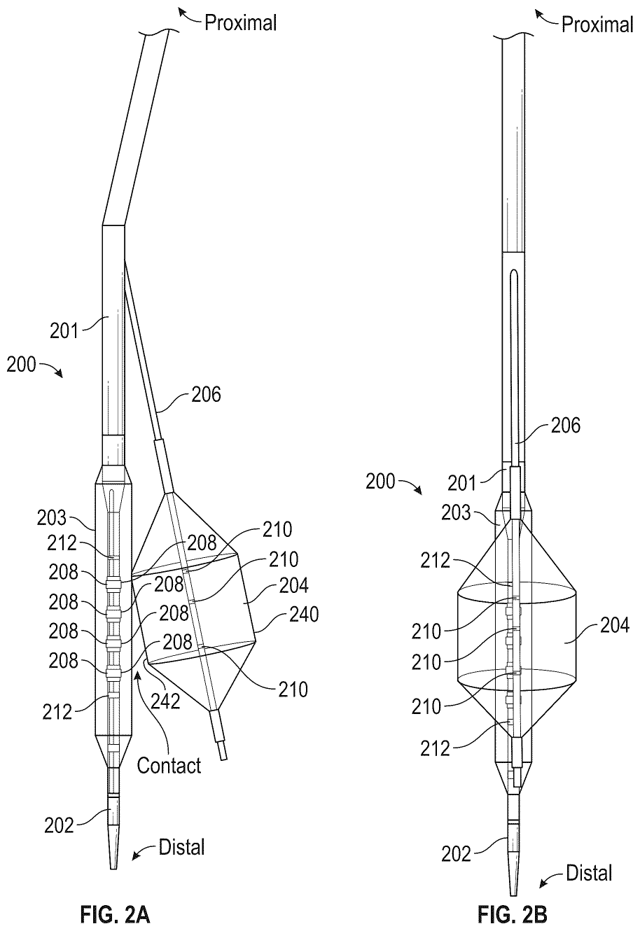

FIGS. 2A and 2B illustrate aspects of an exemplary catheter 200 for generating shock waves within a body lumen that may be used for catheter 10 of FIG. 1. Catheter 200 includes an expandable member 204 that enables a user (e.g., a surgeon or other medical professional) to move at least one shock wave emitter closer to a lesion within a body lumen when expanded. Moving the at least one shock wave emitter closer to a lesion may be advantageous in reducing the attenuation of energy that occurs between the shock wave source and the lesion. Catheter 200 thus enables more effective treatment of lesions within the body by allowing users to impact lesions with relatively more powerful shock waves.

FIG. 2A illustrates a first view of catheter 200. The view shown in FIG. 2A is illustrative of catheter 200 when viewed from a first perspective using radiographic imaging. Catheter 200 includes a catheter body 201 and a first elongate member 202. An enclosure 203 is mounted to the first elongate member 202. Enclosure 203 is optionally also mounted to the catheter body 201. In some examples, the first elongate member 202 forms part of the catheter body 201. The enclosure 203 may be compliant such that it stretches when pressurized, non-compliant such that it does not stretch or stretches minimally when pressurized with a fluid, or semi-compliant. At least one shock wave emitter 208 is carried by the first elongate member and disposed within the enclosure 203. In some examples, the first elongate member 202 may be a distal end portion of the catheter body.

Catheter 200 also includes a second elongate member 206. The second elongate member 206 may be oriented at least partially transverse to the first elongate member 202 and/or the catheter body 201. An expandable member 204 is mounted to the second elongate member 206 and configured to expand to contact the enclosure 203 mounted to the first elongate member 202. The expandable member may be configured to expand when inflated with a fluid. The expandable member 204 can thus be used to push the first elongate member 202 away from the second elongate member 206 and toward a lesion within a body lumen when expanded. In some examples, the expandable member may be a compliant balloon. In some examples, the expandable member 204 and/or elongate member 206 may be relatively shorter in length than elongate member 202 and/or enclosure 203 such that a distal end of the expandable member is positioned proximally of a distal end of the enclosure. In such examples, the expandable member 204 and/or elongate member 206 may be relatively less prone to becoming entrapped on chordae.

When the expandable member 204 is expanded, one side 240 of the expandable member 204 pushes against a wall of a lumen within which the catheter 200 is disposed and the opposite side 242 of the expandable member pushes the first elongate member 202 away from the second elongate member 206 toward a side of the wall of the lumen opposite the side being pushed by the expandable member, which may move the at least one shock wave emitter 208 closer to a lesion within the lumen. The at least one shock wave emitter 208 may be located on an opposite side of the first elongate member 202 from the second elongate member 206 such that it faces the lesion. Positioning the at least one shock wave emitter 208 on an opposite side of the first elongate member 202 from the second elongate member 206 enables relatively more of the shock wave energy emitted from the at least one shock wave emitter 208 to be directed away from the second elongate member (e.g., toward a lesion) than if the at least one shock wave emitter 208 were located on the same side of first elongate member 202 as the second elongate member 206. Further, when expanded, the angle formed between catheter body 201 and second elongate member 206 can be from five to seventy-five degrees (5°-75°), depending on the amount by which each of expandable member 204 and enclosure 203 are inflated, as appropriate to apposition enclosure 203 proximate to a target tissue or lesion.

In some examples, a plurality of shock wave emitters 208 are positioned at respective longitudinal locations along elongate member 202. Some of the shock wave emitters 208 may be circumferentially aligned with one another. Some of the shock wave emitters 208 may be circumferentially offset from one another around the circumference of elongate member 202.

The shock wave emitters 208 may be positioned at any circumferential location of elongate member 208. In some examples, a plurality of shock wave emitters 208 are positioned at the same longitudinal location of elongate member 202 and circumferentially spaced (e.g., by 30 degrees, 45 degrees, 60 degrees, 75 degrees, 90 degrees, 180 degrees, etc.) from one another around the circumference of elongate member 202. Positioning multiple shock wave emitters at the same longitudinal location and spacing them circumferentially from one another can enable generation of constructively interfering shock waves.

Radiopaque markers may be positioned on the catheter 200 to enable a user to determine the position of the shock wave emitter(s) 208 relative to a target lesion and to reorient the catheter 200 prior to expansion of expandable member 204. Thus, a user can ensure that the at least one shock wave emitter 208 is positioned radially inward of the lesion prior to expansion such that the at least one shock wave emitter 208 is pushed toward the lesion when the expandable member 204 is expanded. A first set of radiopaque markers 212 may be positioned on the first elongate member 202. The first set of radiopaque markers 212 may be positioned within enclosure 203. A second set of radiopaque markers 210 may be positioned on the second elongate member 206. The second set of radiopaque markers 210 may be positioned within expandable member 204.

The first set of radiopaque markers 212 and the second set of radiopaque markers 212 may be arranged on the first and second elongate members, 202 and 206, such that the first set of radiopaque markers 212 appears colinear with the second set of radiopaque markers 210 when viewed from a first perspective using radiographic imaging (e.g., as shown in FIG. 2B.). The first set of radiopaque markers 212 and the second set of radiopaque markers 212 may be arranged on the first and second elongate members, 202 and 206, such that the first set of radiopaque markers 212 appears non-colinear with the second set of radiopaque markers 210 when viewed from a second perspective using radiographic imaging (e.g., as shown in FIG. 2A). Arranging the markers such that they appear colinear from a first perspective and non-colinear from a second perspective assists a user in determining how the first elongate member 202 and second elongate member 206 are positioned within the body using radiographic imaging. For instance, a radiographic imaging device (e.g., an X-Ray device) may image the catheter from a first vantage point and a second vantage point (e.g., 90 degrees offset from the first vantage point) to determine the orientation of the device in the body. For instance, a first image may enable a user to narrow the possible orientations of the shock wave emitters to two possible orientations (e.g., based on whether the two sets of radiopaque markers are aligned or offset in the first image). The imaging device may then be rotated by 90 degrees to capture a second image, which may confirm the orientation of the shock wave emitters. As an illustrative example, if the two sets of radiopaque markers are aligned in the first image (e.g., as shown in FIG. 2B), this may indicate that the shock wave emitters are either positioned “above” the expandable member (i.e., closer to the imaging device compared to the expandable member) or “below” the expandable member (i.e., further from the imaging device compared to the expandable member. The second image taken from 90 degrees offset relative to the first image may confirm whether the shock wave emitters were “above” or “below” the expandable member in the first image.

In some examples, the second set of radiopaque markers 210 has a different arrangement than the first set of radiopaque markers 212. For instance, the second set of radiopaque markers 210 may include a different number of markers than the first set of radiopaque markers 212. The second set of radiopaque markers 210 may additionally, or alternatively, include a different a different spacing between respective markers 210 than the first set of radiopaque markers 212. The first set of radiopaque markers 212 may include at least two markers. At least one marker 212 may be positioned proximally of at least one shock wave emitter 208 and at least one marker 212 may be positioned distally of at least one shock wave emitter 208. In some examples, a marker 212 is positioned proximally of a plurality of shock wave emitters 208 and another marker 212 is positioned distally of the plurality of shock wave emitters 208 on the first elongate member. The second set of radiopaque markers 210 may include at least three markers. In some examples, the at least three markers of the second set of radiopaque markers 210 may be evenly spaced along the second elongate member 206. In some examples, the at least three markers of the second set of radiopaque markers 210 may be unevenly spaced along the second elongate member 206.

Arranging the two sets of radiopaque markers differently on the respective elongate members may enable a user to determine which elongate member is closer to a target lesion within a body lumen. For instance, if the set of markers 210, which includes three markers in this example, is positioned closer to the lesion than the set of markers 212, which includes two markers in this example, then a user can determine that the at least one shock wave emitter 208 is not positioned close to the lesion, and vice versa. It should be understood that the different numbers of radiopaque markers described herein is merely exemplary. Any number and/or arrangement of the two sets of radiopaque markers is within the scope of this disclosure.

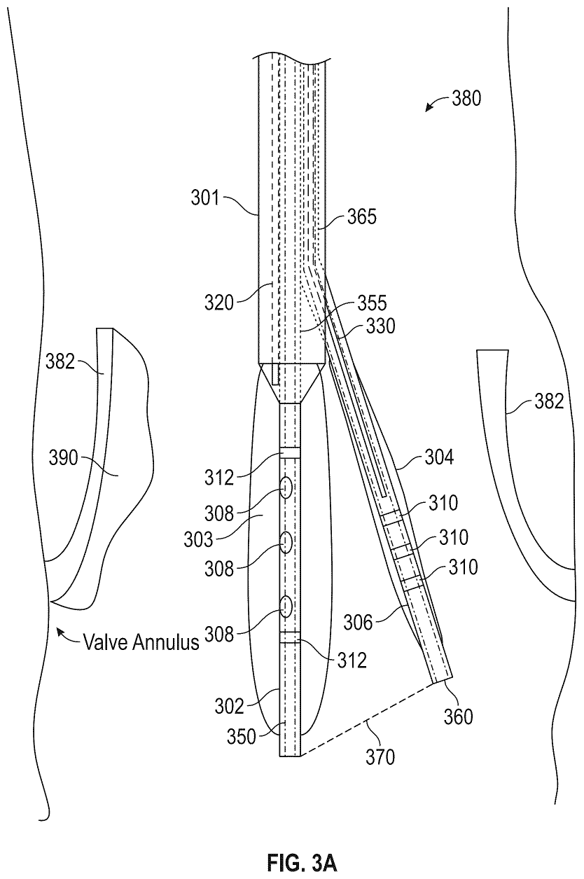

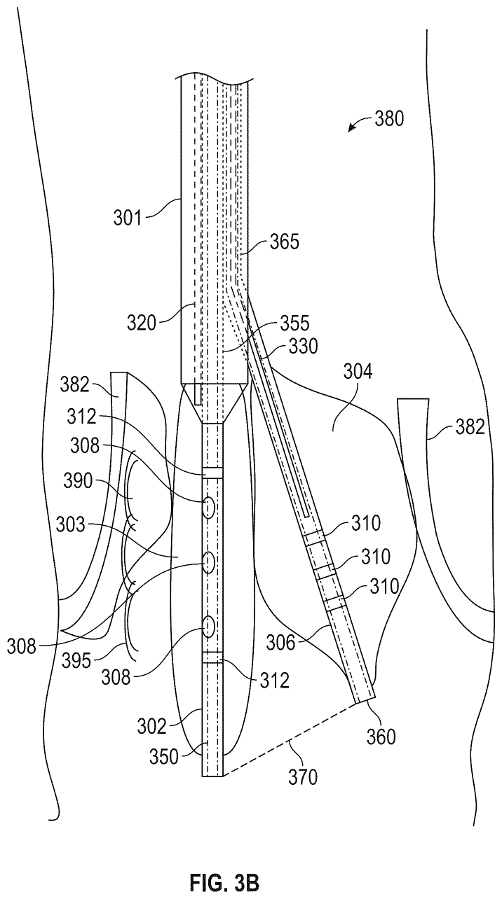

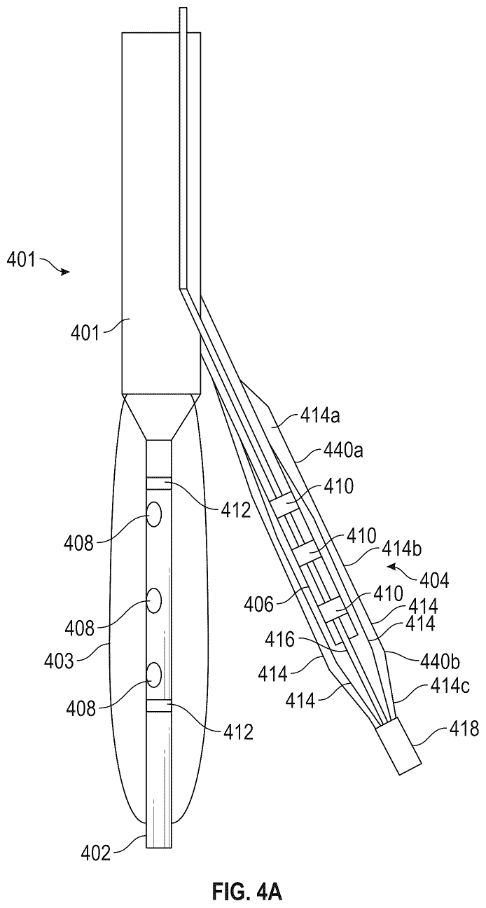

The shock wave generating catheters disclosed herein may be used to treat lesions (e.g., calcifications) in and around cardiac valves, such as the aortic valve and mitral valve. FIGS. 3A and 3B illustrate exemplary aspects of a catheter 300 that may be used for catheter 200 and/or catheter 10 positioned within a cardiac valve 380. FIGS. 3A and 3B also depict internal structure of the catheter 300 that may be included in catheter 200, including guidewire lumens to position the catheters and fluid conduits to fill enclosures of the catheters with fluid. FIGS. 3A and 3B also depict how elongate members, such as elongate member 202, may be attached to a catheter via insertion into lumens of the catheter (e.g., when elongate member 202 is not integral to catheter body 201).

As shown in FIG. 3A, catheter 300 includes a catheter body 301 and a first elongate member 302. A proximal portion of elongate member 302 may be inserted into to a lumen 355 of catheter 300 and extend along at least a portion of catheter body 301 within lumen 355. Alternatively, the elongate member 302 may be inserted from a proximal end of catheter body 301 and extend along the length of the catheter body 301 within lumen 355. An enclosure 303 is mounted to the first elongate member 302. The enclosure 303 may be compliant such that it stretches when pressurized, non-compliant such that it does not stretch or stretches minimally when pressurized with a fluid, or semi-compliant. At least one shock wave emitter 308 is carried by the first elongate member and disposed within the enclosure 303.

Catheter 300 also includes a second elongate member 306. The second elongate member 306 may be connected to the catheter body 301 at a distal portion of the catheter body and may extend distally adjacent to the first elongate member 302. A proximal portion of elongate member 306 may be inserted into a lumen 365 and extend proximally into the catheter body 301. Alternatively, the elongate member 302 may be inserted from a proximal end of catheter body 301 and extend along the length of the catheter body 301 within lumen 365. In some examples, lumen 365 merges with lumen 355 such that elongate member 302 and elongate member 306 extend proximally within the same lumen along at least a portion of catheter body 301. An expandable member 304 is mounted to the second elongate member 306 and configured to expand when filled with a fluid to contact the enclosure 303 mounted to the first elongate member 302.

The catheter 300 may include a first fluid supply conduit 320 for filling enclosure 303 and a second fluid supply conduit 330 for filling expandable member 304. Enclosure 303 and expandable member 304 can thus be filled independently of one another. It may be desirable to leave enclosure 303 filled with a conductive fluid during shock wave treatment while independently deflating expandable member 304 to reposition catheter 300 within cardiac valve 380 (or other body lumen) and then re-expanding (e.g., inflating) expandable member 304, as described further below.

A first set of radiopaque markers 312 may be positioned on the first elongate member 302. The first set of radiopaque markers 312 may be positioned within enclosure 303. A second set of radiopaque markers 310 may be positioned on the second elongate member 306. The second set of radiopaque markers 310 may be positioned within expandable member 304. The first set of radiopaque markers 312 and second set of radiopaque markers 310 may be arranged in any manner described above with reference to FIGS. 2A and 2B and may be used to orient catheter 300 within valve 380.

A guidewire lumen 350 may extend along the catheter body 301 and elongate member 302 to a distal end of the elongate member 302. In some examples, another guidewire lumen 360 extends along catheter body 301 and elongate member 306 to a distal end of elongate member 306. Accordingly, a guidewire may be received into elongate member 302 via guidewire lumen 350 and exit the guidewire lumen 350 at the distal end of elongate member 302 to position the catheter 300 within a body lumen, such as cardiac valve 380. Additionally, or alternatively, a guidewire may be received into elongate member 306 via guidewire lumen 360 and exit the guidewire lumen 360 at the distal end of elongate member 306 to position the catheter 300 within a body lumen, such as cardiac valve 380. In some examples, elongate member 302 and elongate member 306 may be connected via an attachment member 370 at their respective distal ends. In other examples, a distal end of elongate member 302 is free of a distal end of elongate member 306.

During use of catheter 300, a user may insert a guidewire into guidewire lumen 350 and/or 360, and catheter 300 may be advanced within a body lumen to a target treatment site. For instance, catheter 300 may be advanced within a body lumen to a cardiac valve 380, such as a mitral valve or aortic valve. Catheter 300 may be positioned such that elongate member 302 and elongate member 306 are radially inward of and longitudinally aligned with leaflets 382 of valve 380 (or other target treatment site of the valve). Once positioned at valve 380, a user may rotate the catheter 300 to reorient the catheter 300 such the at least one shock wave emitter 308 is positioned relatively closer to a lesion 390, which may be formed on a leaflet 382 or other aspect of the valve 380. A fluid (e.g., conductive fluid) may be introduced into enclosure 303 via fluid conduit 320. Fluid may also be introduced into expandable member 304 via fluid conduit 330 to expand the expandable member 304.

FIG. 3B illustrates the expandable member 304 in an expanded configuration. The expandable member 304 may be expanded such that a portion of the expandable member 304 contacts and pushes against a portion of the body lumen, such as against a leaflet 382, as shown in FIG. 3B. As expandable member 304 is inflated with fluid, it pushes against the enclosure 303, moving enclosure 303 and elongate member 302 away from elongate member 306 and toward the lesion 390. A plurality of shock wave emitters 308 carried by elongate member 302 are thus pushed laterally relatively closer to lesion 390. As shown in the example depicted in FIG. 3B, when the expandable member 304 is in the expanded state, the enclosure 303 may be pressed against a lesion within the lumen 380, such as against lesion 390. After expanding expandable member 304 to move shock wave emitters 308 laterally (e.g., radially) closer to the lesion 390, an energy pulse can be applied to the shock wave emitters 308, resulting in shock waves 395 that impinge on and modify (e.g., crack, fragment, otherwise break up) the lesion 390.

While FIG. 3B depicts a lesion on a valve leaflet 382, a lesion could be formed on any other surface of a body lumen 380. For instance, a lesion could be formed on an inner wall of the lumen 380. The at least one shock wave emitter 308 could be positioned such that it is longitudinally aligned with the lesion on the inner wall of lumen 380. Expandable member 304 could then be expanded until a first portion of expandable member 304 pushes against a portion of the wall of the lumen 380 and a second portion of expandable member 304 pushes enclosure 303 against another portion of the lumen 380 (e.g., 180 degrees around the circumference of lumen 380).

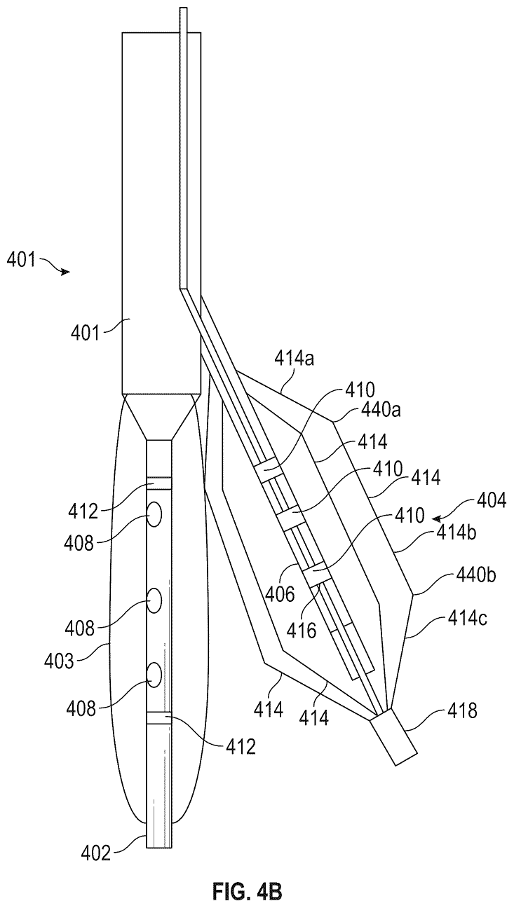

In some examples, an expandable frame (e.g., a mesh frame or wire frame) may be used for the expandable members described herein (e.g., expandable member 204, 304). FIGS. 4A and 4B illustrate aspects of an exemplary catheter 400 that may be used for catheter 10 of FIG. 1 that includes an expandable frame 404. Catheter 400 includes a catheter body 401, a first elongate member 402, and a second elongate member 406. The first elongate member 402 may include any of the aspects described above with reference to elongate member 202 and/or elongate member 302 and may be connected to or form part of a distal portion of the catheter body 401. Elongate member 406 may be connected to the distal portion of catheter body 401 and may be oriented at least partially transverse to elongate member 402. Expandable frame 404 may be connected to elongate member 406. FIG. 4A illustrates expandable member 404 in a collapsed configuration and FIG. 4B illustrates expandable member 404 in an expanded configuration.

The expandable frame 404 may include a plurality of laterally expandable members 414. The laterally expandable members 414 are moveable between a collapsed configuration (FIG. 4A) and an expanded configuration (FIG. 4B) using a moveable shaft 416 connected to expandable frame 404. Moving the moveable shaft 416 proximally toward a proximal end of the catheter 400 may cause the expandable frame 404 to expand laterally. Moving the moveable shaft distally toward a distal end of the catheter 400 may cause the expandable frame 404 to collapse laterally. The expandable frame 404 may be configured to allow a fluid to pass by or through the frame when the frame is expanded.

The moveable shaft 416 may extend along the catheter body 401 (e.g., within a lumen, or along the outer surface of the catheter body 401) such that a user can control the shaft from the proximal end of the catheter. The moveable shaft 416 may be connected to a distal tip 418 at the distal end of the moveable shaft 416. The distal tip 418 may be connected to the expandable frame 404 such that moving the distal tip results in expansion or collapse of the expandable frame 404.

Each of the laterally expandable members 414 may include a proximal region 414a, and a distal region 414c, and a connecting region 414b between the proximal region 414a and the distal region 414c. the proximal region 414a of each expandable member 414 may be connected at its proximal end to elongate member 406. The proximal region 414a may extend distally at an angle transverse to the elongate member 406 to a first joint 440a. The first joint 440a may connect the proximal region 414a to connecting region 414b and may be configured to bend when an axial force is applied to the moveable shaft 416. Connecting region 414b may extend distally from the first joint 440a to a second joint 440b that also may be configured to bend when an axial force is applied to the moveable shaft 416. Connecting region 414b may be configured to remain parallel to elongate member 406 in both the expanded and collapsed configurations. The second joint 440b connects the connecting region 414b to the distal region 414c. Distal region 414c may extend distally from the connecting region 414b at an angle transverse to the elongate member 406. A distal end of the distal region 414c may be connected to a distal tip 418. Accordingly, moving the moveable shaft 416 may move the distal tip 418, and thus expand or collapse the expandable frame 404.

During use of catheter 400, a user may advance catheter 400 within a body lumen to a target treatment site, such as a cardiac valve as shown in FIGS. 3A-3B. At the target treatment site, the user may expand expandable frame 404 from the collapsed configuration (FIG. 4A) to the expanded configuration (FIG. 4B). The expandable members 414 may contact enclosure 403 as the expandable frame 404 is moved to the expanded configuration and push the enclosure 403 and elongate member 402 away from elongate member 406. Pushing the enclosure 403 and elongate member 402 away from elongate member 406 enables a user to move a plurality of shock wave emitters 408 carried by elongate member 402 closer to a lesion within a body lumen.

In various implementations, the laterally extendable members 414 in an expanded configuration can have a distance of from one millimeter to ten millimeters (1-10 mm) from the moveable shaft 416. The extent of expanding the extendable members 414 can be controlled to appropriately position the shock wave emitters 408 proximate to a lesion or tissue. For instance, as moveable shaft 416 is moved proximally, the expandable members 414 may gradually expand with the movement of the moveable shaft 416. Similarly, as moveable shaft 416 is moved distally, the expandable members 414 may gradually collapse with the movement of the moveable shaft 416. Thus, a user can control the extend of expanding the extendable members 414 using the moveable shaft 416.

The first elongate member 402 and second elongate member 406 may carry respective sets of radiopaque markers, including a first set of radiopaque markers 412 on elongate member 402 and a second set of radiopaque markers 410 on elongate member 402. The radiopaque markers, including the first set of radiopaque markers 412 and the second set of radiopaque markers 410 may be arranged in any manner described above (e.g., with reference to FIGS. 2A-2B). The radiopaque markers may be advantageous for orienting to elongate members 402 and 406 within the body prior to shock wave treatment.

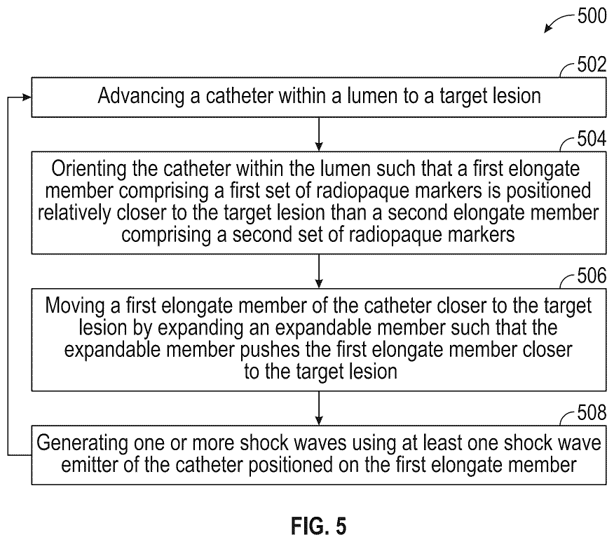

The catheters described herein can be used for treating various lesions (e.g., calcifications, stenosis, chronic total occlusion (CTO), etc.). FIG. 5 illustrates an exemplary method 500 for generating shock waves using the catheters described herein to treat lesions within the body after moving shock wave emitters in close proximity to a lesion.

At block 502, the method 500 includes advancing a catheter within a lumen to a target lesion. The catheter may be, or include any of the features of, any of the catheters described with reference to FIGS. 1-4B. The target lesion may include circumferential calcium, aortic calcification, or other obstructions or concretions. In some examples, the target lesion may include calcified plaques that accumulate on the leaflets and/or annulus of the aortic valve, for instance, as shown in FIGS. 3A and 3B. In other examples, target lesions that can be treated include calcified plaques affecting the mitral value, the tricuspid valve, or the pulmonary valve. Other walls and tissues of the heart with calcified lesions can also be treated.

The catheter may include a catheter body, a first elongate member, and a second elongate member. At least one shock wave emitter may be carried by the first elongate member and disposed within an enclosure mounted to the first elongate member. A first set of radiopaque markers may be positioned on the first elongate member, and a second set of radiopaque markers may be positioned on the second elongate member. The radiopaque markers may be arranged differently on the two elongate members, enabling a user to distinguish between the two elongate members using radiographic imaging to orient the shock wave emitter(s) closer to the target lesion within the lumen (e.g., by rotating the catheter such that the elongate member carrying the shock wave emitter(s) is closer to the lesion).

At block 504, the method 500 includes orienting the catheter such that a first set of radiopaque markers is positioned relatively closer to the target lesion than a second set of radiopaque markers. The catheter may be rotated to orient the first set of radiopaque markers, and thus the at least one shock wave emitter, closer to a target lesion. Due to the different arrangement of the radiopaque markers on the first and second elongate members, radiographic imaging enables a user to determine which elongate member is positioned closer to a target lesion. As the user rotates the catheter within the lumen, the first set of markers and second set of markers will appear colinear in some orientations (e.g., as shown in FIG. 2B) and noncolinear in other orientations (e.g., as shown in FIG. 2A). Additionally, a different arrangement (e.g., different number) of markers may be mounted on the first elongate member than the second elongate member, which may enable a user viewing the catheter within the body using radiographic imaging to determine which portion of an internal wall of a lumen the at least one shock wave emitter is closest to. Thus, the user knows whether the at least one shock wave emitter is positioned proximally to the lesion, or if the catheter needs to be rotated further to position the at least one shock wave emitter for treatment. Orienting the catheter such that a first set of radiopaque markers is positioned relatively closer to the target lesion may position the at least one shock wave emitter such that it is facing the target lesion.

In some examples, prior to orienting the catheter such that the shock wave emitters face the target lesion, an imager (e.g., camera) of the catheter may be used to image the lesion for eccentricity or other morphological characteristics. A user (e.g., physician) may position the catheter based the morphology of the lesion in order to optimize treatment based on the morphology of the lesion.

At block 506, the method 500 may include moving a first elongate member of the catheter closer to the target lesion by expanding an expandable member connected to a second elongate member of the catheter such that the expandable member pushes the first elongate member closer to the target lesion. In some examples, expandable member may be an enclosure configured to expand when inflated with a fluid and push the first elongate member toward the target lesion (e.g., as depicted in FIGS. 2A-3B). Expanding the expandable member may include introducing a fluid into an enclosed region defined by the expandable member. Fluid may be introduced until the expandable member is expanded by a desired amount. In some examples the expandable member may be an expandable frame. Expanding the frame may include moving a movable shaft connected to the expandable fame proximally relative to the frame, for instance as described with reference to FIGS. 4A and 4B.

At block 508, the method 500 includes generating one or more shock waves using at least one shock wave emitter of the catheter. The shock waves may be generated by supplying an energy pulse to a plurality of electrode pairs and/or optical fibers positioned on the catheter that form the at least one shock wave emitter. The shock wave emitters may be configured such that the shock waves propagate outward away from the first elongate member and toward the target lesion through the outer wall of the enclosure. The shock waves may impinge on and modify (e.g., break apart, fracture) a lesion at the target treatment area (e.g., lesion 390 of valve 380 in FIG. 3A). Blocks 502 through 506 may be repeated any number of times.

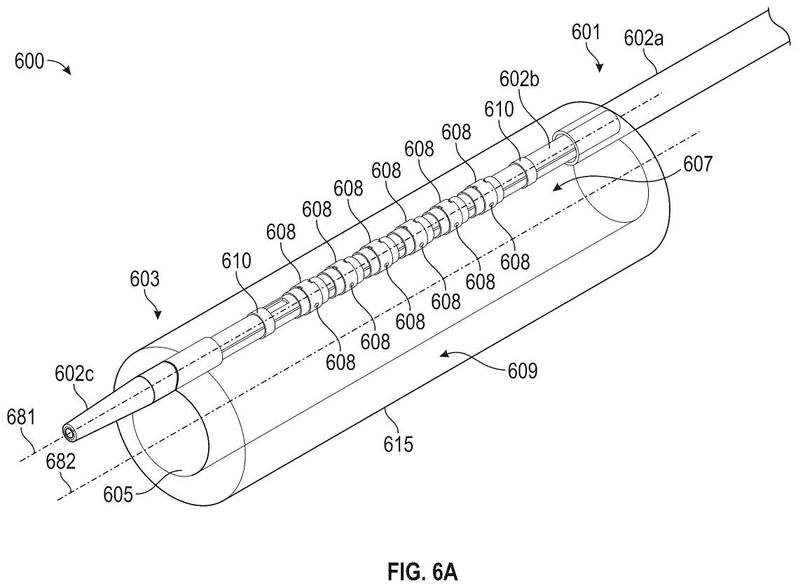

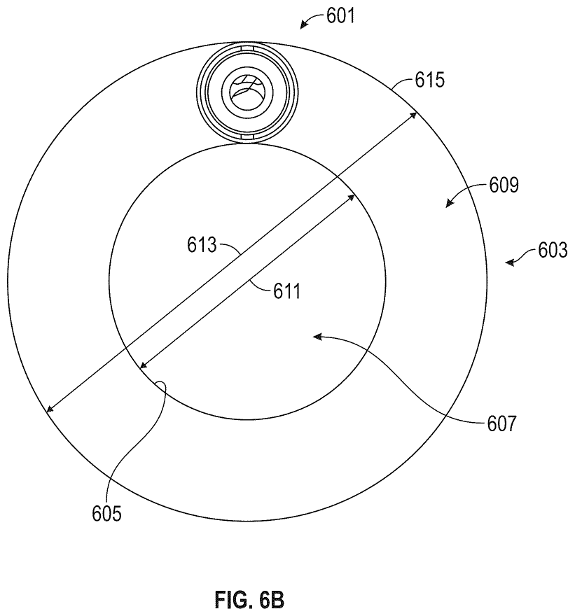

In some examples, it may be desirable to move shock wave emitters closer to a target lesion while minimizing the disruption to blood flow through a body lumen. For instance, it may be advantageous to maintain blood flow through the aortic or mitral valve during treatment. FIG. 6A illustrates an isometric view of aspects exemplary catheter 600 that includes a catheter body 601 and a tube-shaped enclosure 603 sealed to a distal end of the catheter body. The tube-shaped enclosure 603 is configured to enable blood flow to continue within a body lumen while the tube-shaped enclosure 603 is in an expanded configuration.

The tube-shaped enclosure 603 includes an outer cylindrical wall 615, an inner cylindrical wall 605, and a fillable region 609 between the outer cylindrical wall 615 and the inner cylindrical wall 605. The outer cylindrical wall 615 and inner cylindrical wall 605 may form an elongate hollow structure that defines the fillable region 609. The catheter body 601 is positioned at least partially within the fillable region 609 of tube-shaped enclosure 60, and at least one shock wave emitter 608 is disposed on the catheter body 601 within the fillable region 609 between the outer cylindrical wall 615 and the inner cylindrical wall 605.

The fillable region 609 is configured to be filled with a fluid such that the tube-shaped enclosure can be transitioned between a collapsed configuration (not shown) and an expanded configuration (shown in both FIGS. 6A and 6B). The inner cylindrical wall 605 defines an open channel 607 when the tube-shaped enclosure 603 is filled with a fluid (e.g., in the expanded configuration depicted in FIGS. 6A and 6B). The open channel 607 is configured to allow body fluid flowing within a body lumen to flow through the open channel 607 when the catheter 600 is disposed in the lumen and the tube-shaped enclosure 603 is filled with a fluid.

The longitudinal axis 682 of the tube-shaped enclosure extends along open channel 607. The longitudinal axis 681 of the catheter body 601 (and at least one shock wave emitter 608) is offset relative to the tube-shaped enclosure longitudinal axis 682 such that when the tube-shaped enclosure 603 is in the expanded configuration it may become centered in the body lumen and the catheter body 601 may be offset to one side of the lumen.

The at least one shock wave emitter 608 may be configured to emit shock waves toward the outer cylindrical wall 615 when supplied with an energy pulse. The at least one shock wave emitter 608 may be positioned at circumferential location of the catheter body 601 that is closest to the outer cylindrical wall 615. Accordingly, when an energy pulse is supplied to the at least one shock wave emitter 608, shock waves may propagate radially outward from away from the catheter body and through the outer cylindrical wall 615 toward a target lesion. In some examples, the at least one shock wave emitter 608 may be configured to emit shock waves toward an inner cylindrical wall 605 of the tube-shaped enclosure 603. The at least one shock wave emitter 608 may be positioned at circumferential location of the catheter body 601 that is closest to the inner cylindrical wall 605. The at least one shock wave emitter 608 may be positioned at any circumferential location of the catheter body 601. In some examples, a plurality of longitudinally aligned shock wave emitters 608 are circumferentially spaced around the catheter body at the same longitudinal location. For instance, two or more shock wave emitters may be circumferentially spaced apart from one another by 30 degrees, 45 degrees, 60 degrees, 90 degrees, 120 degrees, 160 degrees, 180 degrees, or any amount of spacing therebetween. Positioning a plurality of shock wave emitters at the same longitudinal location but circumferentially spaced from one another enables shock wave emission in a plurality of directions and may promote constructive interference of shock wave energy.

The tube-shaped enclosure 603 can be used to move the catheter body 601 and at least one shock wave emitter 608 closer to the target lesion prior to shock wave treatment. The catheter 600 can be advanced within a body lumen such that the at least one shock wave emitter 608 is longitudinally aligned with the target lesion. The tube-shaped enclosure 603 can then be filled with a fluid to expand the tube-shaped enclosure 603 such that it pushes the catheter body 601 and at least one shock wave emitter 608 laterally (e.g., radially) toward a target lesion. The tube-shaped enclosure 603 may be filled until the outer cylindrical wall 615 contacts an inner wall of a body lumen, thus positioning the at catheter body 601 and at least one shock wave emitter directly adjacent to a target lesion on the inner wall of the lumen.

One or more radiopaque markers 610 may be positioned on the catheter body 601 proximally of and/or distally of the at least one shock wave emitter 608 and may guide a user's placement of the catheter 600 within the body lumen. For instance, the catheter 600 can be positioned such that a distal marker 610 is positioned on one side of a valve annulus and another marker 610 is positioned on the other side of the valve annulus, thus indicating that the at least one shock wave emitter 610 is positioned within the valve annulus.

The catheter body 601 may include a tapered distal end portion 602c, an elongate outer tube 602a located proximally of the tapered distal end portion 602c, and an emitter support structure 602b positioned between the tapered distal end portion 602c and elongate outer tube 602a. A distal end of the tube-shaped enclosure 603 may be sealed to the tapered distal end portion 602c of the catheter body 601, and a proximal end of the tube-shaped enclosure 603 may be sealed to the proximal elongate outer tube 602a. The emitter support structure 602b may be located within the fillable region 609 of the tube-shaped enclosure 603 and may extend proximally within a lumen of the proximal elongate outer tube 602a. The tapered shape of the tapered distal portion 602c of the catheter body may reduce the crossing profile of the catheter relative to a cylindrical shape and may make it easier to navigate the catheter 600 within narrow lumens of the body.

FIG. 6B shows an alternative view of the catheter 600 facing toward the distal end of the catheter 600. The outer cylindrical wall 615 forms an outer diameter 613 of the tube-shaped enclosure 603. The outer diameter 613 when the tube-shaped enclosure is filled with a fluid may be between 10 millimeters and 30 millimeters. The inner-cylindrical wall 605 forms an inner diameter 611 of the tube-shaped enclosure 603. The inner diameter 611 may be at least 6 millimeters.

In some examples, the tube-shaped enclosure 603 is formed from a semi-compliant material or non-compliant material such that it does not stretch or stretches minimally when filled with a fluid. In some examples, the compliance of the outer cylindrical wall 615 may be different than a compliance of the inner cylindrical wall 605. For instance, the outer cylindrical wall may include a compliant material such that it can expand/inflate when filled with a fluid to contact an inner wall of a body lumen. The inner cylindrical wall 605 may be formed from a non-compliant or semi-compliant material such that it does not stretch or stretches minimally when filled with a fluid in order to maintain the open channel 607 when the tube-shaped enclosure is filled with a fluid. The tube-shaped enclosure 603 may include a shape memory material. A shape memory material may be advantageous to ensure the tube-shaped enclosure collapses when a fluid is removed from the enclosure and/or to maintain the shape of the open channel 607.

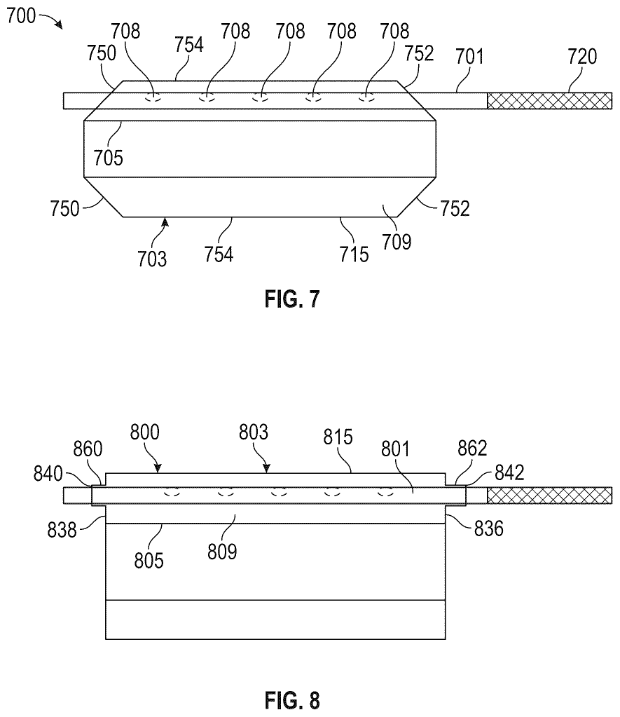

FIG. 7 shows a side view depicting aspects of a catheter 700 including aspects of a tube-shaped enclosure 703 that may be used for the tube-shaped enclosure 603 of FIGS. 6A and 6B. Catheter 700 includes a catheter body 701 and a tube-shaped enclosure 703 configured to enable blood flow to continue within a body lumen while the tube-shaped enclosure 703 is filled with a fluid.

The tube-shaped enclosure 703 includes an outer cylindrical wall 715, an inner cylindrical wall 705, and a fillable region 709 between the outer cylindrical wall 715 and an inner cylindrical wall 705. The inner cylindrical wall 705 defines a channel through which bodily fluid can flow when the fillable region is filled with a fluid. The outer cylindrical wall 715 includes a distal tapered portion 750 and a proximal tapered portion 752. A substantially straight portion 754 extends between the distal tapered portion 750 and a proximal tapered portion 752.

The distal tapered portion 750 may be advantageous for advancing the catheter within a body lumen. For instance, after filling the tube-shaped enclosure 703 with a fluid, the tube-shaped enclosure 703 may not return to its original unfilled size when the fluid is removed. The distal tapered portion 750 reduces the crossing profile of the catheter 700 at the distal end, making it more navigable within small lumens of the body. The proximal tapered portion 752 may be advantageous for the same reason when the catheter 700 is withdrawn from the body and/or moved in the proximal direction within a body lumen.

At least one shock wave emitter 708 is carried by the catheter body 701 and disposed within the fillable region. The at least one shock wave emitter 708 may be positioned at any circumferential location of the catheter body 701, including on a portion of the catheter body 701 facing the outer cylindrical wall 715. When the tube-shaped enclosure is filled with a fluid, the outer cylindrical wall 715 may expand to contact an internal surface of a body lumen. Accordingly, the at least one shock wave emitter 708 can be positioned directly adjacent to and facing the internal wall of the body lumen.

It may be advantageous to enable a user to rotate the catheter 700 such that the at least one shock wave emitter can be oriented toward a lesion within a body lumen before the tube-shaped enclosure 703 is filled with a fluid. A braided shaft 720 (e.g., formed from stainless steel, nitinol, etc.) may be disposed on or form part of a proximal portion of the catheter body 701. The braided shaft 720 may reinforce the catheter body 701 such that it is relatively easier for a user to rotate the catheter body 701 within the body lumen.

FIG. 8 shows a side view depicting aspects of another catheter 800 including aspects of a tube-shaped enclosure 803 that may be used for the tube-shaped enclosure 603 of FIGS. 6A and 6B and/or FIG. 7. Catheter 800 includes a catheter body 801 and a tube-shaped enclosure 803 configured to enable blood flow to continue within a body lumen while the tube-shaped enclosure 803 is filled with a fluid.

The tube-shaped enclosure 803 includes an outer cylindrical wall 815, an inner cylindrical wall 805, and a fillable region 809 between the outer cylindrical wall 815 and an inner cylindrical wall 805. A distal portion of the catheter body 801 is positioned within the fillable region 809 of the tube-shaped enclosure 803. The tube-shaped enclosure 803 includes a proximal opening 842 in a proximal wall 836 and a distal opening 852 in a distal wall 838 of the enclosure. The proximal opening 842 and distal opening 852 provide access into the fillable region of the tube-shaped enclosure 803.

During assembly, the distal portion of the catheter body 801 can be inserted into the tube-shaped enclosure 803 via the proximal opening 842 and may exit the tube-shaped enclosure 803 via distal opening 840. A proximally extending cylindrical wall 862 defining the proximal opening and a distally extending cylindrical wall 860 defining the distal opening may be sealed to the catheter body 801 to form a sealed enclosure configured to be filled with a fluid. The proximally extending cylindrical wall 862 and distally extending cylindrical wall 860 may thus ease assembly of the tube-shaped enclosure 803 to the catheter body 801.

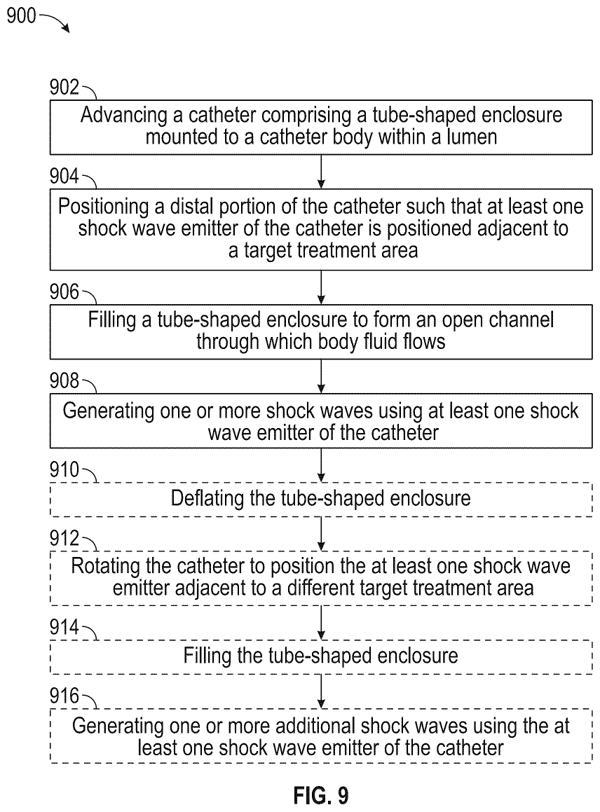

Catheters 600-800 may be used to position shock wave emitters closer to a lesion within the body while allowing bodily fluid to continue flowing through a body lumen undergoing treatment. FIG. 9 illustrates an exemplary method 900 for positioning a shock wave generating catheter closer to a target treatment area using a tube-shaped enclosure that forms an open channel to allow continued blood flow during treatment.

At block 902, the method 900 may include advancing a catheter including a tube-shaped enclosure mounted to a catheter body within a lumen. At block 904, the method 900 may include positioning a distal portion of the catheter such that at least one shock wave emitter of the catheter is positioned adjacent to a target treatment area. The target treatment area may be a lesion (e.g., calcified tissue buildup) within a body lumen. In some examples, the target treatment area may be a region of a valve, such as an aortic valve, mitral valve, or other cardiac valve. An example of such a valve is illustrated in FIGS. 3A and 3B. The distal portion of the catheter may be positioned across the valve annulus such that at least one shock wave emitter of the catheter is positioned adjacent to a valve leaflet (e.g., leaflet 382 of FIG. 3). The catheter may include radiopaque markers (e.g., bands 610 shown in FIG. 6A) to aid in positioning the catheter across the valve annulus.

At block 906, the method 900 may include filling a tube-shaped enclosure. Filling the tube-shaped enclosure may form an open channel through which body fluid flows and may move the at least one shock wave emitter closer to the target treatment area. The at least one shock wave emitter may be disposed on a portion of the catheter located within a fillable region of the tube-shaped enclosure that expands laterally outward when the enclosure is filled. Accordingly, filling the enclosure moves the shock wave emitters laterally outward toward a target treatment area of the lumen (e.g., a calcification on leaflets 382. When filled, an outer wall of the tube-shaped enclosure may contact the leaflets/annulus (e.g., leaflets 382). An inner wall of the tube-shaped enclosure forms an open channel for blood to pass through the center of the balloon (e.g., channel 607 of catheter 600).

At block 908, the method 900 may include generating one or more shock waves using at least one shock wave emitter of the catheter. The shock waves may be generated by supplying an energy pulse to a plurality of electrode pairs and/or optical fibers positioned on the catheter. The shock wave emitters may be configured such that the shock waves propagate outward away from the catheter body and toward the target lesion through the outer wall of the tube-shaped enclosure. The shock waves may impinge on and modify (e.g., break apart, fracture) a lesion at the target treatment area (e.g., valve 380 of FIG. 3A).

At block 910, the method 900 may include deflating the tube-shaped enclosure. After generating shock waves to treat a first lesion, the tube-shaped enclosure may be deflated so that the catheter can be repositioned to treat a different lesion and/or different area of the same lesion. At block 912, the method 900 may include rotating the catheter to position the at least one shock wave emitter adjacent to a different target treatment area. For instance, the catheter may originally be oriented such that the at least one shock wave emitter is facing a first leaflet of a valve (e.g., the leaflet 382 on the left side of FIG. 3A). The catheter may be rotated such that the at least one shock wave emitter faces the second leaflet (e.g., the leaflet 382 on the right side of FIG. 3A). At block 914, the method 900 may include filling the tube-shaped enclosure, and at block 916, the method 900 may include generating one or more additional shock waves using the at least one shock wave emitter of the catheter. Method 900 may be repeated any number of times until treatment is completed.

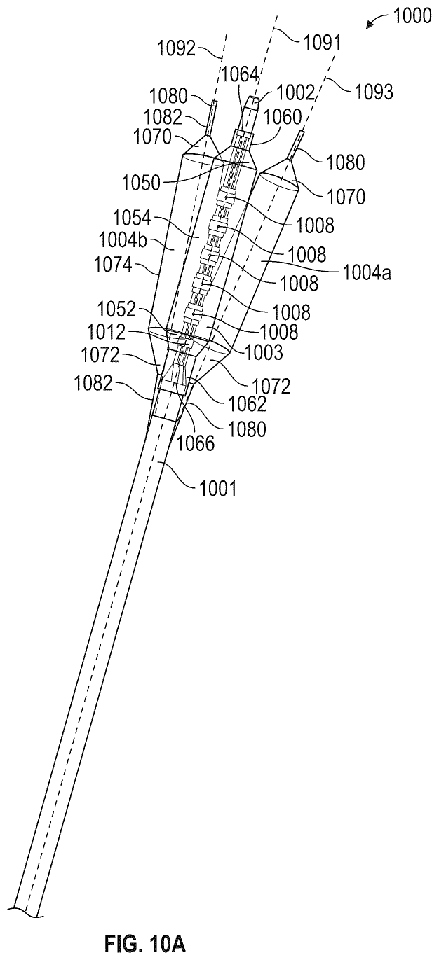

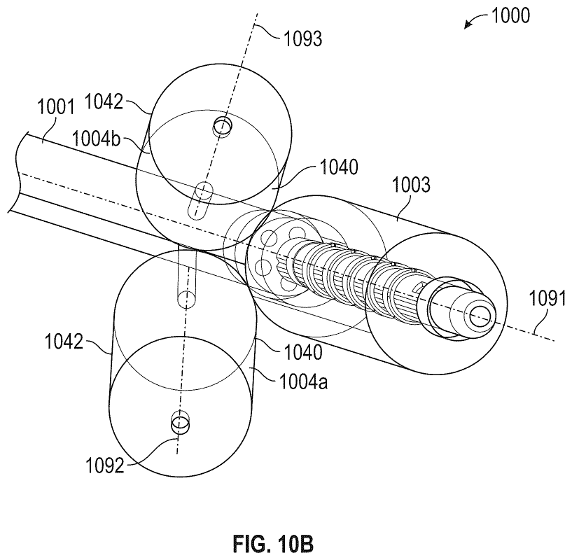

FIGS. 10A and 10B illustrate aspects of an exemplary shock wave catheter 1000 that may be used for catheter 10 of FIG. 1. Catheter 1000 includes a plurality of expandable members including expandable member 1004a and expandable member 1004b, that enable a user (e.g., a surgeon or other medical professional) to move at least one shock wave emitter 1008 closer to a lesion within a body lumen, such as a valve, when expanded. Catheter 1000 is configured such that the at least one shock wave emitter 1008 can positioned closely to a lesion within a valve (such as the valve depicted in FIGS. 3A and 3B) without relying on typical positioning methods such as steering or centering with occlusion. Catheter 1000 may also be configured such that blood flow is not fully occluded when the plurality of expandable members, including expandable member 1004a and expandable member 1004b, are expanded. Catheter 1000 thus enables more effective treatment of lesions within the body by allowing users to impact lesions with relatively more powerful shock waves.

FIG. 10A illustrates a first view of catheter 1000. Catheter 1000 includes a catheter body 1001 and an elongate member 1002. An enclosure 1003 is mounted to the elongate member 1002. Enclosure 1003 is optionally also mounted to the catheter body 1001. In some examples, the elongate member 1002 forms part of the catheter body 1001. The enclosure 1003 may be compliant such that it stretches when pressurized, non-compliant such that it does not stretch or stretches minimally when pressurized with a fluid, or semi-compliant. At least one shock wave emitter 1008 is carried by the elongate member 1002 and disposed within the enclosure 1003. In some examples, the elongate member 1002 may be a distal end portion of the catheter body. In some examples, the elongate member 1002 may extend within a lumen along at least a portion of the length of catheter body 1001.

Catheter 1000 includes a plurality of expandable members, including expandable member 1004a and expandable member 1004b connected to the catheter body 1001. The expandable members 1004a and 1004b are configured to expand to contact the enclosure 1003 and push the enclosure 1003, elongate member 1002, and the at least one shock wave emitter 1008 laterally toward a lesion. FIG. 10B illustrates a second view of catheter 1000. As depicted in FIG. 10B, a first portion 1042 of each of the expandable members 1004a and 1004b may push against a surface of a body lumen or valve (such as the valve depicted in FIGS. 3A and 3B), and a second portion 1040 of each respective expandable member 1004a and 1004b may push against the enclosure 1003 to move the enclosure 1003 laterally toward another portion of the lumen or valve. The plurality of expandable members, including expandable member 1004a and expandable member 1004b, may be configured to expand when filled with a fluid. In some examples, the plurality of expandable members, including expandable member 1004a and expandable member 1004b, are formed from a compliant material such that they stretch when pressurized, a non-compliant material such that it they do not stretch or stretch minimally when pressurized with a fluid, or they may be semi-compliant. The expandable members 1004a and 1004b may be mounted to respective elongate members. Expandable member 1004a may be mounted to an elongate member 1080 and expandable member 1004b may be mounted to another elongate member 1082 (it should be understood that, in some examples, expandable members 1004a and 1004b may instead be empty balloons without any elongate members extending within their interior). At least one fluid conduit may extend along elongate members 1002, 1080, and/or 1082 to fill the enclosure 1003, expandable member 1004a, and expandable member 1004b with a fluid. In some examples, the enclosure 1003, expandable member 1004a, and/or expandable member 1004b may be fillable independently of one another.

The catheter body 1001 and elongate member 1002 extend along a longitudinal axis 1091. Expandable member 1004a and elongate member 1080 may extend away from the elongate member 1002 and longitudinal axis 1091 in a first direction and expandable member 1004b and elongate member 1082 may extend away from elongate member 1002 and longitudinal axis 1091 in a second direction.

Expandable member 1004a and elongate member 1080 extend away from the catheter body along a different longitudinal axis 1093 that may be oriented at least partially transverse to longitudinal axis 1091. Expandable member 1004b and elongate member 1082 extend away from the catheter body along another longitudinal axis 1092 that may be oriented at least partially transverse to longitudinal axis 1091 and longitudinal axis 1093. In some examples, longitudinal axis 1093 is offset from longitudinal axis 1091 and extends distally away from longitudinal axis 1091 at an angle. In some examples, longitudinal axis 1092 is offset from longitudinal axis 1091 and extends distally away from longitudinal axis 1091 at an angle. In some examples, longitudinal axis 1093 is offset from longitudinal axis 1092 and extends distally away from longitudinal axis 1092 at an angle. In some examples, expandable members 1004a and 1004b may not be mounted to elongate members. For example, expandable members 1004a and 1004b may be balloons that extend directly from the catheter body 1001. A proximal end of expandable member 1004a and expandable member 1004b may be connected to a fluid conduit in catheter body 1001 at their respective proximal ends. In some examples, expandable member 1004a and expandable member 1004b have the same outer diameter when filled with a fluid. In some examples, enclosure 1003 has the same outer diameter as expandable member 1004a and expandable member 1004b when each are filled with a fluid.

The at least one shock wave emitter 1008 may be located on an opposite side of the first elongate member 1002 from the expandable members 1004a and 1004b such that the at least one shock wave emitter 1008 is relatively closer to the lesion than it would be if positioned on the side of the first elongate member 1002 that is closest to the expandable members 1004a and 1004b. In some examples, however, a plurality of shock wave emitters 1008 may be spaced circumferentially from one another at one or more longitudinal positions of the elongate member 1002. In some examples, a plurality of shock wave emitters 1008 are positioned at respective longitudinal locations along elongate member 1002. Some of the shock wave emitters 1008 may be circumferentially aligned with one another. Some of the shock wave emitters 1008 may be circumferentially offset from one another around the circumference of elongate member 1002. The shock wave emitters 1008 may be positioned at any circumferential location of elongate member 1008. In some examples, a plurality of shock wave emitters 1008 are positioned at the same longitudinal location of elongate member 1002 and circumferentially spaced (e.g., by 30 degrees, 45 degrees, 60 degrees, 75 degrees, 90 degrees, 180 degrees, or any other degree of separation between 1 degree and 180 degrees) from one another around the circumference of elongate member 1002. Positioning multiple shock wave emitters at the same longitudinal location and spacing them circumferentially from one another enables generation of constructively interfering shock waves. For instance, two shock wave emitters positioned 90 degrees apart from one another may respectively generate shock waves that constructively interfere at a location between the two shock wave emitters (e.g., 45 degrees), resulting in an a relatively more powerful combined shock wave. In some examples, the at least one shock wave emitter includes a conductive emitter band that has an elongate slot formed into the emitter band serving as an electrode of at least one shock wave emitter, for instance, as described in detail in U.S. application Ser. No. 19/319,045, filed on Sep. 4, 2025, the content of which is incorporated herein by reference in its entirety.

Enclosure 1003 may include a distal tapered portion 1050 and a proximal tapered portion 1052. A substantially straight cylindrical portion 1054 extends between the distal tapered portion 1050 and a proximal tapered portion 1052. The distal tapered portion 1050 may be advantageous for advancing the catheter within a body lumen. For instance, after filling the enclosure 1003 with a fluid, the enclosure 1003 may not return to its original unfilled size when the fluid is removed. The distal tapered portion 1050 reduces the crossing profile of the catheter 1000 at the distal end, making it more navigable within small lumens of the body. The proximal tapered portion 1052 may be advantageous for the same reason when the catheter 1000 is withdrawn from the body and/or moved in the proximal direction within a body lumen.

Enclosure 1003 may include a proximal opening 1066 in a proximal wall 1062 and a distal opening 1064 in a distal wall 1060. The elongate member 1002 may extend into the enclosure 1003 via the proximal opening 1066 and a distal end of the elongate member 1002 may exit the enclosure 1003 via the distal opening 1064. The proximal wall 1062 and the distal wall 1060 may form cylindrical tubes extending from the proximal tapered portion 1052 and the distal tapered portion 1050, respectively. The proximal wall 1062 and the distal wall 1060 may be sealed to a respective proximal and distal portion of the elongate member 1002.

The plurality of expandable members, including expandable member 1004a and expandable member 1004b may each include a distal tapered portion 1070 and a proximal tapered portion 1072. A substantially straight cylindrical portion 1074 may extend between the distal tapered portion 1070 and the proximal tapered portion 1072. The distal tapered portion 1072 may be advantageous for advancing the catheter within a body lumen. For instance, after filling the respective expandable members 1004a and 1004b with a fluid, the expandable members may not return to their original unfilled size when the fluid is removed. The distal tapered portion 1070 reduces the crossing profile of the expandable members at their respective distal ends, making them more navigable within small lumens of the body. The proximal tapered portion 1052 may be advantageous for the same reason when the catheter 1000 is withdrawn from the body and/or moved in the proximal direction within a body lumen.

In some examples, one or more radiopaque markers 1012 are mounted to elongate member 1002. One or more radiopaque markers 1012 may additionally or alternatively be mounted to elongate member 1080 and/or elongate member 1082. The one or more radiopaque markers 1012 may be configured to enable a user to position the at least one shock wave emitter 1008 in proximity to a lesion within the body. The one or more radiopaque markers 1012 may be arranged differently on each of the elongate members (1002, 1080, and 1082) to enable a user to distinguish between the respective elongate members and determine the location of the at least one shock wave emitter 1008 relative to a lesion in the body. For instance, a first number of radiopaque markers 1012 may be positioned on elongate member 1002, a second number (different from the first) of radiopaque markers 1012 may be positioned on elongate member 1080, and a third number (different from the first and second) may be positioned on elongate member 1082. As another examples, radiopaque markers 1012 on elongate member 1002 may be spaced by a first distance, radiopaque markers 1012 on elongate member 1080 may be spaced by a second distance (different from the first), and radiopaque markers 1012 on elongate member 1082 may be spaced by a third distance (different from the first and second).

During use of catheter 1000, a user may position the catheter 1000 across a valve, such as the aortic valve. Expandable member 1004a, expandable member 1004b, and enclosure 1003 may be positioned in the three valve commissures (e.g., of the aortic valve). One or more radiopaque markers 1012 mounted to elongate member 1002 may aid in the positioning of the device. For instance, a user may position the device such that a first radiopaque marker 1012 is positioned on a first side of the valve (e.g., on a first side of the valve annulus) and a second radiopaque marker 1012 is positioned on a second side of the valve. Once positioned, Expandable member 1004a, expandable member 1004b, and enclosure 1003 may be filled with a fluid, and one or more shock waves may be generated using the at least one shock wave emitter 1008 to modify a lesion in or near the valve (or other body lumen). Expandable member 1004a, expandable member 1004b, and enclosure 1003 may then be deflated, and a user may rotate the catheter 1000 to reposition the at least one shock wave emitter 1008. For instance, the user may reposition each of the expandable member 1004a, expandable member 1004b, and enclosure 1003 in a different commissure such that the at least one shock wave emitter 1008 can be used to treat a lesion in a different commissure. Expandable member 1004a, expandable member 1004b, and enclosure 1003 may then be re-inflated, and one or more additional shock waves may be generated. This process can be repeated to treat a plurality of different lesions or different portions of lesions. For instance, the user may iterate the process above to treat each commissure of the aortic valve. Additionally, or alternatively, treatment of the left ventricular outflow tract (LVOT) may be performed by advancing the device into the ventricle and applying one or more shock waves using the at least one shock wave emitters 1008. It should be understood that catheter 1000 can be used in contexts other than treating lesions in the commissures of the aortic valve, for instance, in the ventricles, in the vasculature, or in other body lumens. Catheter 1000 enables one or more shock wave emitters to be positioned closer to a lesion (e.g., within a valve commissure) without fully occluding the valve or requiring a steerable device. Additionally, catheter 1000 may be positioned using fluoroscopy only instead of requiring more advanced imaging techniques.

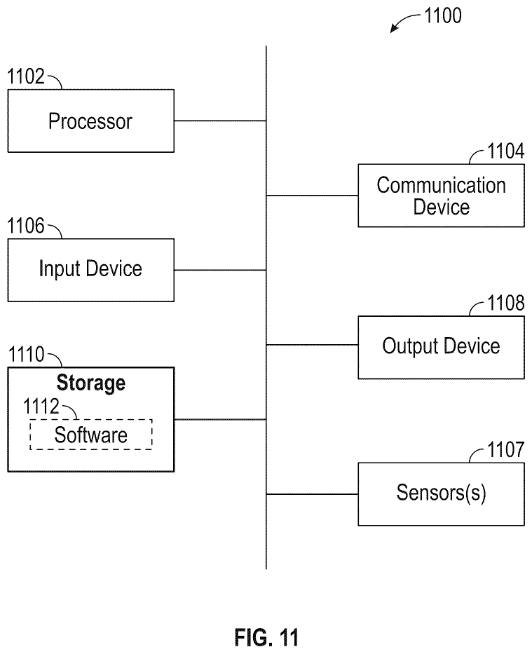

FIG. 11 depicts an exemplary computing system 1100 which may form part of the system 100 described above and may be used for controlling one or more aspects of the devices described herein and/or for performing various steps of the methods described herein, in accordance with one or more examples of the disclosure. System 1100 can be a host computer connected to a network. System 1100 can be a client computer or a server. As shown in FIG. 11, system 1100 can be any suitable type of microprocessor-based device, such as a personal computer, workstation, server, or handheld computing device (i.e., a portable electronic device) such as a phone or tablet. The device can include, for example, one or more processors 1102, input device 1106, sensor device 1107, output device 1108, storage 1110, and communication device 1104. Input device 1106 and output device 1108 can generally correspond to those described above and can either be connectable or integrated with the computer.

Input device 1106 can be any suitable device that provides directed input, such as a touch screen, keyboard or keypad, mouse, or voice-recognition device, in other words, input or directions provided or initiated by a user. Sensor device 1107 can be one or more of any suitable sensor devices, such as a pressure sensor, a thermal sensor, an electrical sensor (e.g., current, voltage, resistance, and/or impedance sensors), or a visualization element. Output device 1108 can be any suitable device that provides output, such as a touch screen, haptics device, or speaker. Storage 1110 can be any suitable device that provides storage, such as an electrical, magnetic, or optical memory, including a RAM, cache, hard drive, or removable storage disk. Communication device 1104 can include any suitable device capable of transmitting and receiving signals over a network, such as a network interface chip or device. The components of the computer can be connected in any suitable manner, such as via a physical bus or wirelessly.

Sensor devices 1107 can provide feedback to an operator using system 1100 by measuring parameters in the surrounding environment and thereby indicating a status of a shock wave catheter device such as catheter 10 connected to computing system 1100, and further providing for guidance on what further steps the operator may decide to implement a shock wave catheter device such as catheter 10 connected to computing system 1100. For example, in implementations where sensor devices 1107 include pressure sensors, a slight decrease in pressure may indicate success at cracking a calcified lesion, due to the fact that an expandable member (e.g., enclosure 120) surrounding shock wave emitters is able to further expand without changing the volume of fluid within the expandable member. Further, a significant decrease in pressure may indicate a rupture failure mode where the expandable member has lost seal and fluid volume, and thus guiding toward withdrawal of the device (e.g., device 10). In implementations where the sensor devices include a visualization element, an operator of a catheter device such as catheter 10 may be able to more clearly understand where the catheter is located relative to a target lesion or anatomy, prior to, during, and after delivering therapy.

In some embodiments, sensor device 1107 includes surface electrodes of an electrocardiograph to synchronize a shock wave to the “R” wave for treating vessels near the heart. Sensor device 1107 may include an R-wave detector and a controller to control the high voltage switch. Mechanical shocks can stimulate heart muscle and could lead to an arrhythmia. While it is unlikely that shock waves of such short duration as contemplated herein would stimulate the heart by synchronizing the pulses (or bursts of pulses) with the R-wave, an additional degree of safety may be provided when used on vessels of the heart or near the heart. In implementations where shock waves are generated from open unenclosed emitters, synchronization to the R-wave would significantly improve the safety against unwanted arrhythmias.

Processor(s) 1102 can be any suitable processor or combination of processors, including any of, or any combination of, a central processing unit (CPU), field programmable gate array (FPGA), and application-specific integrated circuit (ASIC). Software 1112, which can be stored in storage 1110 and executed by processor 1102, can include, for example, the programming that embodies the functionality of the present disclosure (e.g., as embodied in the devices as described above). Software 1112 can also be stored and/or transported within any non-transitory computer-readable storage medium for use by, or in connection with, an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1110, that can contain or store programming for use by, or in connection with, an instruction execution system, apparatus, or device. Software 1112 can also be propagated within any transport medium for use by, or in connection with, an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by, or in connection with, an instruction execution system, apparatus, or device. The transport-readable medium can include, but is not limited to, an electronic, magnetic, optical, electromagnetic, or infrared wired or wireless propagation medium.

System 1100 may be connected to a network, which can be any suitable type of interconnected communication system. The network can implement any suitable communication protocols and can be secured by any suitable security protocols. The network can comprise network links of any suitable arrangement that can implement the transmission and reception of network signals, such as wireless network connections, T1 or T3 lines, cable networks, DSL, or telephone lines. System 1100 can implement any operating system suitable for operating on the network. Software 1112 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

System 1100 may be configured to selectively control the delivery of energy from one or more of energy sources (e.g., a voltage pulse generator or a light energy source) to one or more acoustic energy emitters (e.g., a forward-firing emitter, a radially-firing emitter, an unenclosed emitter, or an enclosed emitter) depending on input from input device 1106.

System 1100 may be configured to tune the energy properties of energy delivered to one or more of the above-described emitters based on tissue properties received from sensor device 1107. Tissue properties may include lesion tissue type (e.g., calcific, thrombic, fibrotic), lesion morphology (e.g., thickness, length, eccentricity).

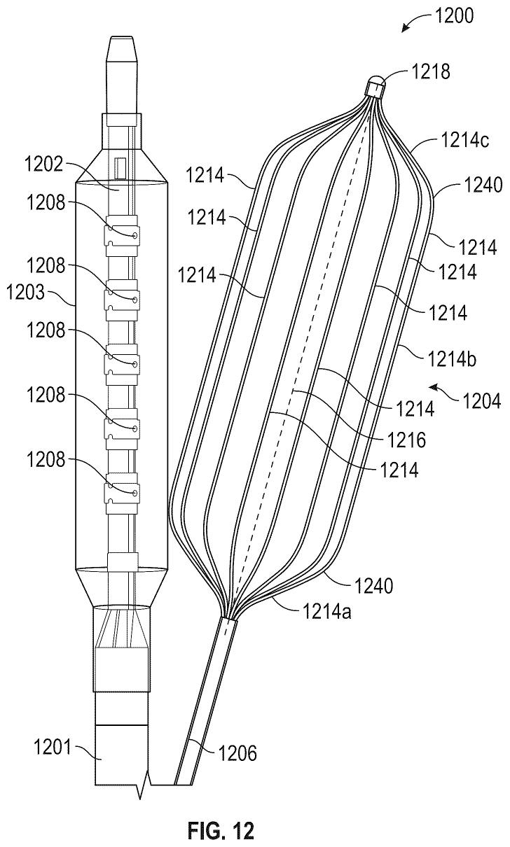

As discussed with reference to FIGS. 4A and 4B, in some examples, an expandable frame (e.g., a mesh frame or wire frame) may be used for the expandable members described herein (e.g., expandable member 204, 304). FIG. 12 illustrates aspects of an exemplary catheter 1200 that includes an exemplary expandable frame. Catheter 1200 may be used for catheter 10 of FIG. 1. Catheter 1200 includes an expandable frame 1204 that may be used to move one or more shock wave emitters 1208 closer to a treatment site (e.g., within a heart valve). Use of an expandable frame, such as expandable frame 1204 (and/or expandable frame 404 described above), to position shock wave emitters 1208 closer to a treatment side may be advantageous in allowing blood flow through the heart valve during use. When the catheter 1200 is located in a blood vessel and the expandable frame is expanded, blood can flow through the expandable frame 1204. Accordingly, during use, catheter 1200 may not occlude or block blood flow through a vessel or heart valve undergoing treatment.

Catheter 1200 includes a catheter body 1201, a first elongate member 1202, and a second elongate member 1206. The first elongate member 1202 may include any of the aspects described above with reference to elongate member 202 and/or elongate member 302 and may be connected to or form at least part of a distal portion of the catheter body 1201. Elongate member 1206 may be connected to the distal portion of catheter body 1201 and may be oriented at least partially transverse to elongate member 1202. Expandable frame 1204 may be connected to elongate member 1206. FIG. 12 illustrates expandable member 1204 in an expanded configuration. The expandable member may be adjustable (e.g., manually by a user) to collapse the expandable member to a collapsed configuration (e.g., similar to the collapsed configuration of the expandable member depicted in FIG. 4B).

The expandable frame 1204 may include a plurality of laterally expandable members 1214. The laterally expandable members 1214 may be moved between the expanded configuration shown in FIG. 12 and a collapsed configuration, such as in a manner similar to that described with reference to FIGS. 4A and 4B and/or via any user-engageable mechanism for expanding and contracting the expandable frame 1204. For instance, catheter 1200 may include a moveable shaft 1216. Moving the moveable shaft 1216 proximally toward a proximal end of the catheter 1200 may cause the expandable frame 1204 to shorten and expand laterally. Moving the moveable shaft distally may cause the expandable frame 1204 to lengthen and collapse laterally.

The moveable shaft 1216 may extend along the catheter body 1201 (e.g., within a lumen, or along the outer surface of the catheter body 1201) to a proximal end of the catheter such that a user can control the shaft from the proximal end of the catheter. The moveable shaft 1216 may be connected at its distal end to a distal tip 1218. The distal tip 1218 may be connected to the expandable frame 1204 such that moving the distal tip results in expansion or collapse of the expandable frame 1204. In some examples, the catheter 1200 does not include a moveable shaft 1216 for expanding or collapsing the expandable members 1214. The expandable members 1214 may configured to self-expand, for instance, by forming the expandable members 1214 using a pre-shaped memory material.

Any, or all, of the laterally expandable members 1214 may include a proximal region 1214a, a distal region 1214c, and a connecting region 1214b between the proximal region 1214a and the distal region 1214c. The proximal region 1214a of each expandable member 1214 may be connected at its proximal end to elongate member 1206. In the expanded configuration, the proximal region 1214a may extend distally at an angle transverse to the elongate member 1206. The proximal region 1214a may extend distally to a first bending region 1240. The first bending region 1240 may connect the proximal region 1214a to connecting region 1214b and may be configured to bend when an axial force is applied to the moveable shaft 1216. Connecting region 1214b may extend distally from the first bending region 1240 to a second bending region 1240 that also may be configured to bend when an axial force is applied to the moveable shaft 1216. Connecting region 1214b may be configured to remain parallel to elongate member 1206 in both the expanded and collapsed configurations. The second bending region 1240 connects the connecting region 1214b to the distal region 1214c. Distal region 1214c may extend distally from the connecting region 1214b. The distal region 1214c may extend distally at an angle transverse to the elongate member 1206 in the expanded configuration. A distal end of the distal region 1214c may be connected to a distal tip 1218. Accordingly, moving the moveable shaft 1216 may move the distal tip 1218, and thus expand or collapse the expandable frame 1204.

During use of catheter 1200, a user may advance catheter 1200 within a body lumen to a target treatment site, such as a cardiac valve as shown in FIGS. 3A-3B, with the expandable frame 1204 in a collapsed configuration. At the target treatment site, the user may expand expandable frame 1204 from the collapsed configuration to the expanded configuration (e.g., the configuration shown in FIG. 12). The expandable members 1214 may contact enclosure 1203 as the expandable frame 1204 is moved to the expanded configuration and push the enclosure 1203 and elongate member 1202 away from elongate member 1206. Pushing the enclosure 1203 and elongate member 1202 away from elongate member 1206 enables a user to move a plurality of shock wave emitters 1208 carried by elongate member 1202 closer to a lesion within a body lumen.

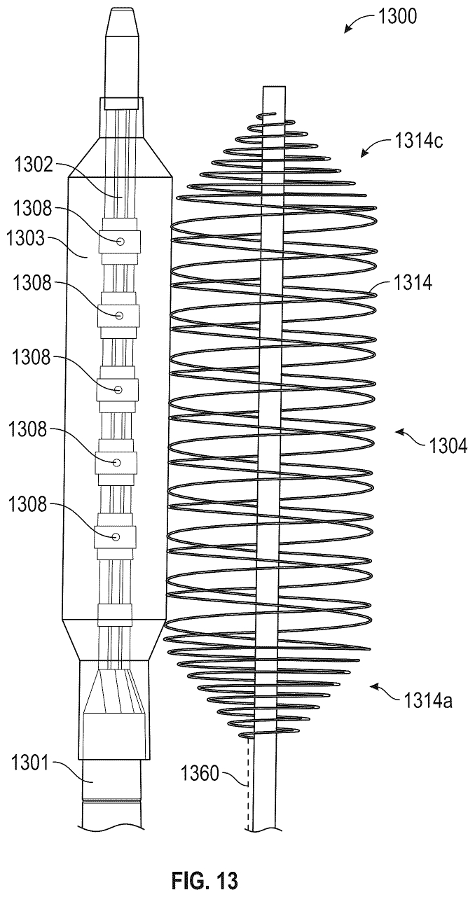

It should be understood that the expandable frame 1204 depicted in FIG. 12 is meant to be exemplary. Other types (e.g., different shapes, sizes, etc.) of expandable frames are within the scope of this disclosure. For instance, FIG. 13 illustrates aspects of an exemplary catheter 1300, which has a differently shaped expandable frame 1304, and may be used for catheter 10 of FIG. 1. Catheter 1300 includes a catheter body 1301, a first elongate member 1302, and a second elongate member 1306. The first elongate member 1302 may include any of the aspects described above with reference to elongate member 202 and/or elongate member 302 and may be connected to or form part of a distal portion of the catheter body 1301. Elongate member 1306 may be connected to the distal portion of catheter body 1301 and may be oriented at least partially transverse to elongate member 1302.

Expandable frame 1304 may be connected to elongate member 1306. The expandable frame 1304 may be used to move one or more shock wave emitters 1308 closer to a treatment site (e.g., within a heart valve). The expandable frame 1304 may be adjustable (e.g., manually by a user) to collapse the expandable frame 1304 from an expanded configuration depicted in FIG. 13 to a collapsed configuration. In the collapsed configuration, a maximum diameter of the expandable frame 1304 may be smaller relative to the maximum diameter of the expandable frame depicted in FIG. 13. In some implementations, the expandable frame 1304 can be formed of a material having a shape-memory or spring force such that when deployed at a treatment site, the expandable frame automatically expands to an operational size. In other implementations, a translatable control member 1360 can be connected to the expandable frame such that an operator can manually change the configuration of the expandable frame between expanded and collapsed configurations. Translation of the control member can be rotational around the circumference of the device, longitudinal along the length of the device, or a combination thereof.

In various implementations, the expandable frame 1304 in an expanded configuration can have a diameter of from one millimeter to fifteen millimeters (1-15 mm). In some examples, the expanded diameter of the expandable frame is between 25% and 150% of the diameter of enclosure 1303. In some examples, the expanded diameter of the expandable frame 1304 is at least two times the diameter of the enclosure 1303, at least three times the diameter of the enclosure 1303, at least four times the diameter of the enclosure 1303, at least five times the diameter of the enclosure 1303, at least six times the diameter of the enclosure 1303, at least seven times the diameter of the enclosure 1303, at least eight times the diameter of the enclosure 1303, at least nine times the diameter of the enclosure 1303, or at least ten times the diameter of the enclosure 1303. The extent of expanding the expandable frame 1314 can be controlled to appropriately position the shock wave emitters 1308 proximate to a lesion or tissue. When the catheter 1300 is located in a blood vessel and the expandable frame is expanded, blood can flow through the expandable frame 1304.

The expandable frame 1304 illustrated in FIG. 13 includes a coiled member 1314. The coiled member of expandable frame 1304 may include a tapered proximal portion 1314a and a tapered distal portion 1314c. The tapered proximal portion 1314a and tapered distal portion 1314c may have respective average diameters that are smaller than a portion of the coiled member 1314 extending between the tapered proximal portion 1314a and tapered distal portion 1314c. The tapered proximal portion 1314a and tapered distal portion 1314c may make it easier for a user to advance and retract the catheter 1300 within the body.

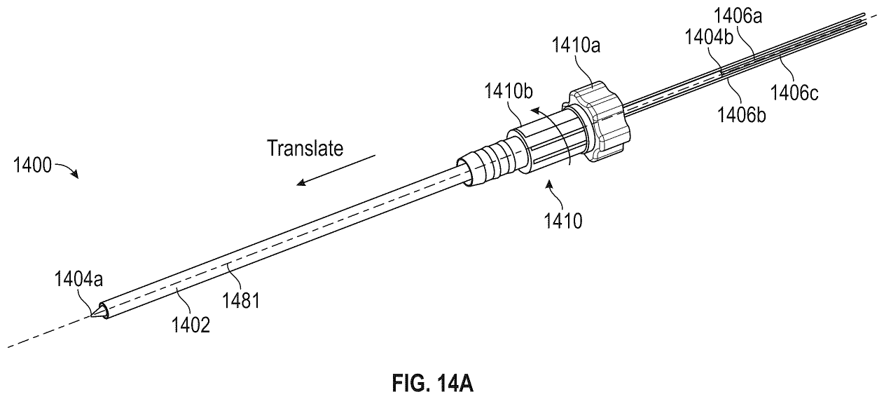

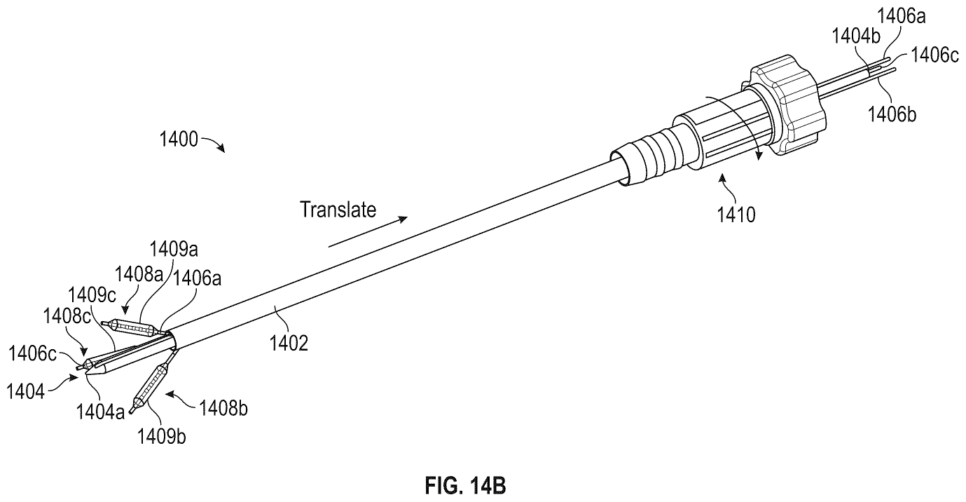

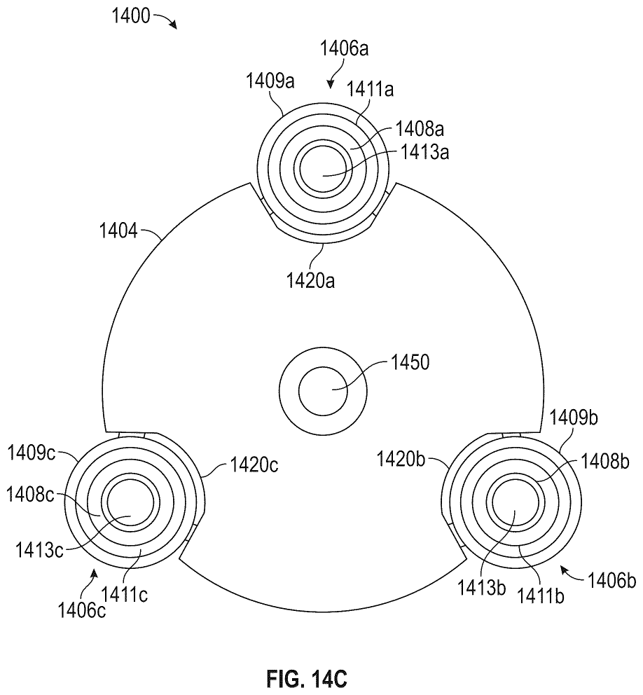

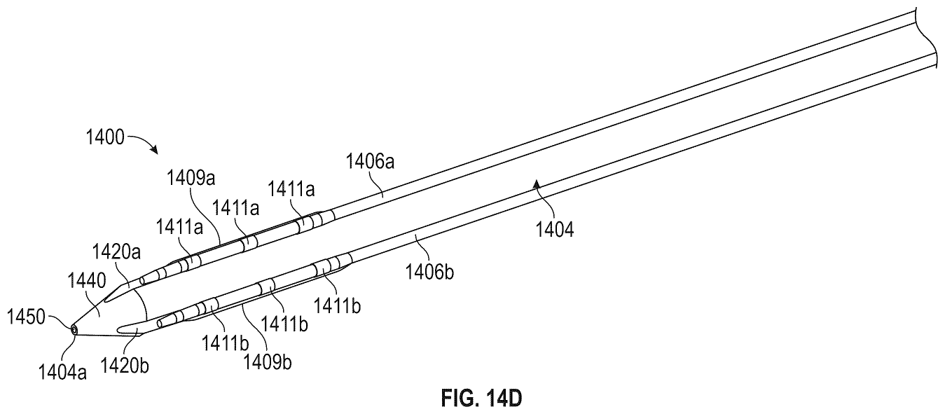

FIGS. 14A-14D illustrate aspects of an exemplary catheter 1400 that may be used for catheter 10 of FIG. 1. Catheter 1400 includes laterally moveable shock wave emitter support members including shock wave emitter support members 1406a, 1406b, and 1406c that can be used to position shock wave emitters closer to lesions, for instance, between cusps of an aortic valve. Catheter 1400 may include an inner elongate member 1404 and an outer elongate member 1402. The inner elongate member 1404 and outer elongate member 1402 may be configured such that they can be translated relative to one another. For instance, in some examples, the inner elongate member 1404 may be positioned radially inward of the outer elongate member 1402 and configured such that a user can slide the inner elongate member 1404 distally and/or proximally relative to the outer elongate member 1402. The shock wave emitter support members 1406a, 1406b, and 1406c may be positioned between the inner elongate member 1404 and the outer elongate member 1402. The shock wave emitter support members 1406a, 1406b, and 1406c may be mounted to the inner elongate member 1404 and may be configured translate together with the inner elongate member 1404 relative to the outer elongate member 1402. When the inner elongate member 1404 is in an extended position (e.g., as illustrated in FIG. 14B), the shock wave emitter support members 1406a, 1406b, and 1406c may move outwardly away from the inner elongate member 1404. When the inner elongate member is translated proximally to the retracted position, the shock wave emitter support members 1406a, 1406b, and 1406c may move inward toward the inner elongate member 1404 (e.g., as the outer elongate member 1402 pushes the shock wave emitter support members 1406a, 1406b, and 1406c toward the inner elongate member 1404).

In some examples, the outer elongate member 1402 may be positioned radially outward of the shock wave emitter support members and configured to translate between an extended position and a retracted position relative to the inner elongate member 1404. When the outer elongate member is in the extended position, the shock wave emitter support members may be positioned at least partially within the outer elongate member (e.g., a sheath), and the outer elongate member may restrict the outward movement of the emitter support members. The catheter 1400 may thus maintain a relatively low profile while the catheter is navigated within the vasculature and the outer elongate member is in the extended position (and/or when the inner elongate member 1404 is in the retracted position within the outer elongate member). When the catheter 1400 is positioned at a treatment site, the outer elongate member can be retracted to a retracted position (or the inner elongate member 1404 can be translated distally to an extended position) to allow the emitter support members to move outwardly away from the longitudinal axis of the catheter, and a user can position a respective emitter support member adjacent to a plurality of target treatment sites for shock wave treatment.

Catheter 1400 includes an inner elongate member 1404 and a plurality of emitter support members, including emitter support members 1406a, 1406b, and 1406c, positioned along the inner elongate member 1404. Catheter 1400 also includes an outer elongate member 1402 positioned radially outward of the inner elongate member 1404. The outer elongate member 1402 extends along at least a portion of the inner elongate member 1404. The outer elongate member 1402 may circumscribe/surround at least a portion of the inner elongate member 1404. In some examples, the inner elongate member may be translatable (e.g., slidable) proximally or distally relative to the outer elongate member 1402 between an extended position of the inner elongate member 1404, an example of which is shown in FIG. 14B, and a retracted position of the inner elongate member 1404, an example of which is shown in FIG. 14A. In some examples, the outer elongate member 1402 may be translatable (e.g., slidable) proximally or distally relative to the inner elongate member 1404 between an extended position, an example of which is shown in FIG. 14A, and a retracted position, an example of which is shown in FIG. 14B.

Sliding the outer elongate member distally toward a distal end 1404a of the inner elongate member 1404 (or sliding the inner elongate member 1404 proximally) may cover at least a portion of the emitter support members 1406a, 1406b, and 1406c, positioning a distal portion 1408a, 1408b, and 1408c of emitter support members 1406a, 1406b, and 1406c, respectively, within the outer elongate member 1402, as illustrated in FIG. 14A. The outer elongate member 1402 covering the distal portion 1408a, 1408b, and 1408c of emitter support members 1406a, 1406b, and 1406c may prevent the emitter support members from moving outwardly away from inner elongate member 1404 and a longitudinal axis 1481 of the catheter 1400. Sliding the outer elongate member proximally toward a proximal end 1404b of the inner elongate member 1404 (or sliding the inner elongate member 1404 distally) may expose respective distal portions 1408a, 1408b, and 1408c of the emitter support members 1406a, 1406b, and 1406c, as illustrated in FIG. 14B. When the respective distal portions 1408a, 1408b, and 1408c are uncovered, each respective distal portion 1408a, 1408b, and 1408c may move outwardly away from the inner elongate member 1404. The emitter support members 1406a, 1406b, and 1406c may each carry one or more shock wave emitters at its respective distal portion 1408a, 1408b, and 1408c, as described in further detail below. When the distal portions 1408a, 1408b, and 1408c move outwardly, the one or more shock wave emitters may be moved closer to a target lesion (such as a calcification within or near a cardiac valve).

At least the distal portions 1408a, 1408b, and 1408c of emitter support members 1406a, 1406b, and 1406c may be configured to curve or bend outwardly from the inner elongate member 1404 when the outer elongate member 1402 is not covering the distal portions 1408a, 1408b, and 1408c. At least a portion of the emitter support members 1406a, 1406b, and 1406c, including the distal portions 1408a, 1408b, and 1408c, may be formed from or comprise a shape memory material, such as pre-shaped nitinol. In some examples, a shape memory material, such as pre-shaped nitinol wire, may be inserted into a respective lumen of the emitter support members 1406a, 1406b, and 1406c, including along at least a portion of the distal portions 1408a, 1408b, and 1408c. When outer elongate member 1402 is positioned in the retracted position such that the distal portions 1408a, 1408b, and 1408c are uncovered, the pre-shaped material may cause the distal portions 1408a, 1408b, and 1408c to move outwardly away from inner elongate member 1404 (as shown in FIG. 14B).

Catheter 1400 may include a locking member 1410 configured such that a user can selectively lock and unlock the outer elongate member 1402 to the inner elongate member 1404. The locking member 1410 may be configured to lock the outer elongate member 1402 to the inner elongate member 1404 when the locking member 1410 is rotated in a first direction and to unlock the outer elongate member 1402 from the inner elongate member 1404 when the locking member 1410 is rotated in a second direction. In some examples, a first portion 1410a of the locking member 1410 may be rotated relative to a second portion 1410b of the locking member 1410 to lock and unlock the locking member 1410. Thus, the locking member, when locked, may prevent translation of the outer elongate member 1402 relative to the inner elongate member 1404. The locking member 1410 may be a Tuohy Burst or other locking mechanism.

FIG. 14C depicts additional detail of the distal end of the inner elongate member 1404, including additional aspects of the emitter support members 1406a, 1406b, and 1406c. The inner elongate member 1404 may include a plurality of grooves, including groove 1420a, 1420b, and 1420c, that extend along the inner elongate member 1404. The emitter support members, including emitter support members 1406a, 1406b, and 1406c may each be positioned within a respective groove of the plurality of grooves 1420a, 1420b, and 1420c. For instance, emitter support member 1406a may be positioned within groove 1420a, emitter support member 1406b may be positioned within groove 1420b, and emitter support member 1406c may be positioned within groove 1420c. A portion (e.g., a proximal portion) of one or more of the emitter support members 1406a-1406c may be glued or otherwise fixed to a respective one of the grooves 1420a-1420c. Inner elongate member may also include a guidewire lumen 1450 extending along the length of the inner elongate member (e.g., from proximal end 1404b to distal end 1404a. During use, the catheter 1400 may be advanced along a guidewire positioned within the guidewire lumen 1450.

At least one shock wave emitter may be positioned on each of the emitter support members. The at least one shock wave emitter may include any of the features of the shock wave emitters disclosed herein, for instance, as described with reference to shock wave emitters 16 of FIG. 1. At least one shock wave emitter 1411a may be positioned on the distal portion 1408a of emitter support member 1406a, at least one shock wave emitter 1411b may be positioned on the distal portion 1408b of emitter support member 1406b, and at least one shock wave emitter 1411c may be positioned on the distal portion 1408c of emitter support member 1406c. The shock wave emitter or emitters provided on each of the emitter support members may be positioned within a respective enclosure mounted to each of the emitter support members. The enclosure may include one or more of the features described with reference to enclosure 18, expandable member 204, enclosure 203, expandable member 304, enclosure 303, tube-shaped enclosures 603, 703, 803, expandable members 1004a-1004c, and/or enclosures 1203 and 1303.

A first enclosure 1409a may be mounted to emitter support member 1406a. Enclosure 1409a may enclose at least part of the distal portion 1408a of emitter support member 1406a and the at least one shock wave emitter 1411a. A second enclosure 1409b may be mounted to emitter support member 1406b. Enclosure 1409b may enclose at least part of the distal portion 1408b of emitter support member 1406b and the at least one shock wave emitter 1411b. A third enclosure 1409c may be mounted to emitter support member 1406c. Enclosure 1409c may enclose at least part of the distal portion 1408c of emitter support member 1406c and the at least one shock wave emitter 1411c. One or more of the enclosures mounted to the emitter support members, including enclosure 1409a, 1409b, and 1409c, may be configured to expand when filled or inflated with a conductive fluid. One or more of the enclosures 1409a, 1409b, and 1409c may be formed from a semi-compliant material or non-compliant material such that it does not stretch or stretches minimally when filled with a fluid.

In some examples, a plurality of shock wave emitters may be positioned on one or more of the emitter support members. FIG. 14D illustrates a detailed perspective view illustrating aspects of the inner elongate member 1404 and emitter support members 1406a and 1406b. A first plurality of shock wave emitters 1411a may be positioned along emitter support member 1406a within enclosure 1409a. In some examples, emitters 1411a may be positioned at equal distances from one another along the length of emitter support member 1406a. In some examples, one or more of the plurality of emitters 1411a may be positioned relatively closer to one another one or more other emitters 1411a of the plurality of emitters 1411a along the length of emitter support member 1406a. A second plurality of shock wave emitters 1411b may be positioned along emitter support member 1406b within enclosure 1409b. In some examples, emitters 1411b may be positioned at equal distances from one another along the length of emitter support member 1406b. In some examples, one or more of the plurality of emitters 1411b may be positioned relatively closer to one another one or more other emitters 1411b of the plurality of emitters 1411b along the length of emitter support member 1406b. A plurality of shock wave emitters 1411c may similarly be positioned on emitter support member 1406c (not shown in FIG. 14D). It should be understood that any number of emitter support members may be provided on catheter 1400 and any number of shock wave emitters may be positioned on each of the respective emitter support members.

As discussed above, a shape memory material, such as pre-shaped nitinol wire, may be inserted into a respective lumen of the emitter support members 1406a, 1406b, and 1406c, including along at least a portion of the distal portions 1408a, 1408b, and 1408c. Returning to FIG. 14C, a respective lumen is depicted extending along each of emitter support members 1406a, 1406b, and 1406c. A first lumen 1413a extends along emitter support member 1406a, a second lumen 1413b extends along emitter support member 1406b, and a third lumen 1413c extends along emitter support member 1406c. A respective pre-shaped member, such as a pre-shaped nitinol wire, can be inserted into one or more of the lumens, which may cause the emitter support members 1406a, 1406b, and 1406c to move (e.g., curve/bend) outwardly from at least a portion of the inner elongate member 1404 when the portion of the inner elongate member 1404 is not covered by the outer elongate member 1402.

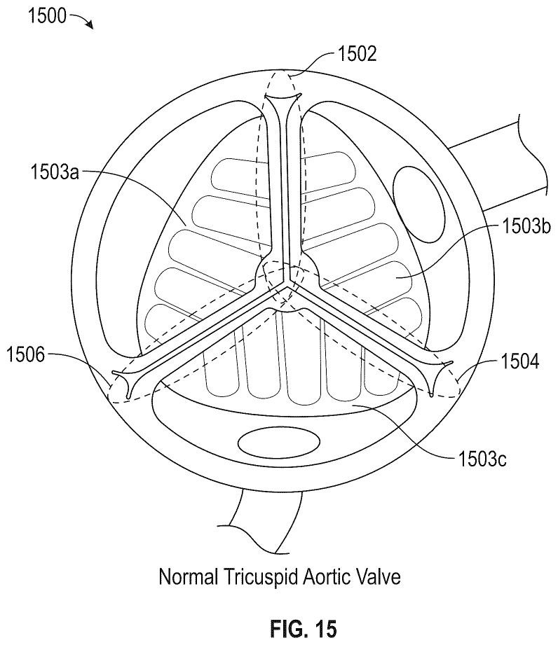

As described throughout, the various catheters disclosed herein may be used to treat lesions (e.g., calcified buildup) in or around cardiac valves, such as the aortic valve. FIG. 15 illustrates aspects of an aortic valve 1500, which the catheters disclosed herein may be used to treat. Valve 1500 is a tricuspid valve that includes three leaflets 1503a, 1503b, and 1503c. During use of catheter 1400, the distal portions 1408a, 1408b, and 1408c of emitter support members 1406a, 1406b, and 1406c, respectively, may be positioned between the leaflets of the valve 1500. For instance, a first emitter support member may be positioned in region 1502 of valve 1500, a second emitter support member may be positioned in region 1504 of valve 1500, and a third emitter support member may be positioned in region 1506 of valve 1500.

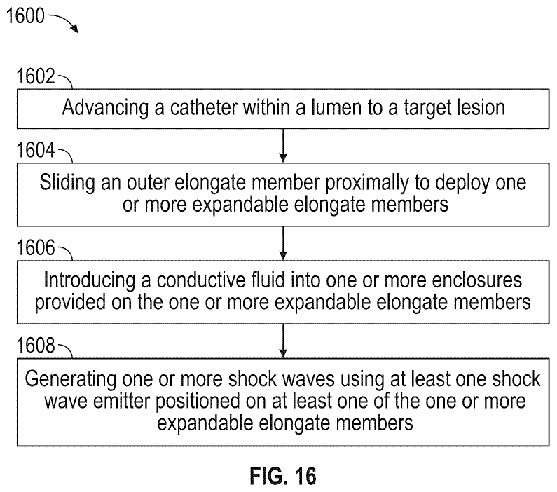

FIG. 16 illustrates a flowchart representing steps of a method 1600 for generating shock waves using catheter 1400 of FIGS. 14A-E, for instance, to treat lesions formed between leaflets of the valve 1500. At block 1602, a user (e.g., a healthcare professional) can advance a catheter (e.g., catheter 1400) over a guidewire to a target treatment site, for instance, into the left ventricle or other cardiac valve, such as the valve 1500 depicted in FIG. 15. At block 1604, the user may slide/translate an outer elongate member of the catheter proximally relative to an inner elongate member of the catheter (for instance, with reference to catheter 1400, from the extended position depicted in FIG. 14A to the retracted position depicted in FIG. 14B). In some examples, prior to sliding the outer elongate member of the catheter proximally relative to the inner elongate member of the catheter, the user may unlock a locking member of the catheter (e.g., locking member 1410) to unlock the outer elongate member from the inner elongate member. The user may unlock the locking member by rotating the locking member. Sliding the outer elongate member of the catheter proximally relative to the inner elongate member of the catheter may expose a distal portion of a plurality of emitter support members (e.g., emitter support members 1406a-1406c). Exposing the distal portion of the plurality of emitter support members may enable the distal portion of the plurality of emitter support members to move outwardly from a longitudinal axis of the catheter and/or from an inner elongate member of the catheter toward the target treatment area. In examples where the target treatment site is a cardiac valve (e.g., valve 1500), the user may manipulate the catheter to place each of the distal portions of the catheter between leaflets of the valve (e.g., within one of regions 1502, 1504, and 1506 of valve 1500). At block 1606, the method 1600 may include introducing an electrically conductive fluid into one or more enclosures provided on the one or more expandable elongate members. The conductive fluid may be introduced into each of the enclosures (e.g., enclosures 1409a-1409c of catheter 1400) independently of one or more of the remaining enclosures or may be introduced into all of the enclosures simultaneously. At block 1608, one or more shock waves may be generated using at least one shock wave emitter positioned on at least one of the one or more emitter support members. Following treatment, the user may translate/slide the outer elongate member distally relative to emitter support members and/or the inner elongate member such that it covers the distal portions of the emitter support members pushing the emitter support members inwardly toward the longitudinal axis of the catheter and/or the inner elongate member.

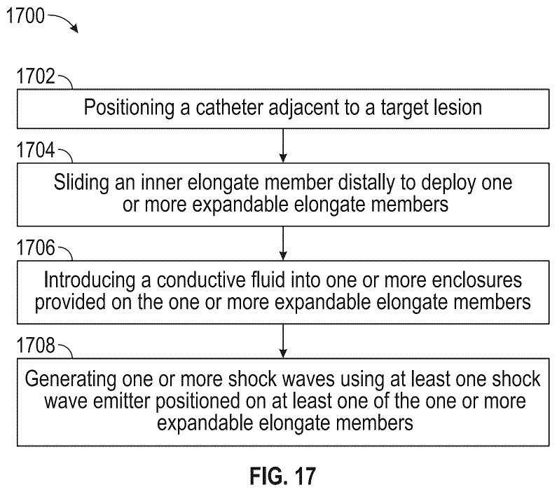

FIG. 17 illustrates a flowchart representing steps of a method 1700 for generating shock waves using catheter 1400 of FIGS. 14A-E, for instance, to treat lesions formed between leaflets of the valve 1500. At block 1702, a user (e.g., a healthcare professional) can advance a catheter (e.g., catheter 1400) over a guidewire to a target treatment site, for instance, into the left ventricle or other cardiac valve, such as the valve 1500 depicted in FIG. 15. At block 1704, the user may slide/translate an inner elongate member of the catheter distally relative to an outer elongate member of the catheter (for instance, with reference to catheter 1400, from the retracted position depicted in FIG. 14A to the extended position depicted in FIG. 14B).

In some examples, prior to sliding the inner elongate member of the catheter distally relative to the outer elongate member of the catheter, the user may unlock a locking member of the catheter (e.g., locking member 1410) to unlock the outer elongate member from the inner elongate member. The user may unlock the locking member by rotating the locking member. Sliding the inner elongate member of the catheter distally relative to the outer elongate member of the catheter may expose a distal portion of a plurality of emitter support members (e.g., emitter support members 1406a-1406c). Exposing the distal portion of the plurality of emitter support members may enable the distal portion of the plurality of emitter support members to move outwardly from a longitudinal axis of the catheter and/or from an inner elongate member of the catheter toward the target treatment area.

In examples where the target treatment site is a cardiac valve (e.g., valve 1500), the user may manipulate the catheter to place each of the distal portions of the catheter between leaflets of the valve (e.g., within one of regions 1502, 1504, and 1506 of valve 1500). At block 1706, the method 1700 may include introducing an electrically conductive fluid into one or more enclosures provided on the one or more expandable elongate members. The conductive fluid may be introduced into each of the enclosures (e.g., enclosures 1409a-1409c of catheter 1400) independently of one or more of the remaining enclosures or may be introduced into all of the enclosures simultaneously. At block 1708, one or more shock waves may be generated using at least one shock wave emitter positioned on at least one of the one or more emitter support members. Following treatment, the user may translate/slide the outer inner member proximally relative to the outer elongate member such that it covers the distal portions of the emitter support members pushing the emitter support members inwardly toward the longitudinal axis of the catheter and/or the inner elongate member.

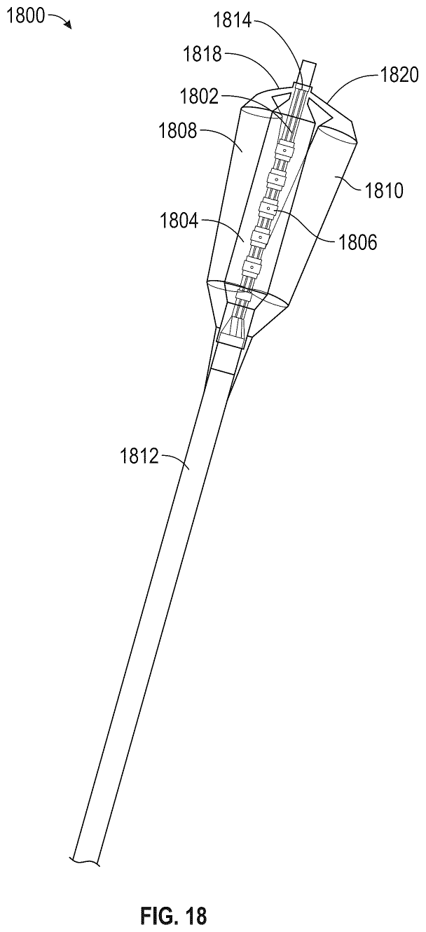

FIG. 18 illustrates aspects of an exemplary shock wave catheter 1800 that may be used for catheter 10 of FIG. 1. Catheter 1800 may share one or more features in common with catheter 1000 of FIGS. 10A-10B. Catheter 1800 includes a plurality of expandable members including expandable member 1808 and expandable member 1810, that enable a user (e.g., a surgeon or other medical professional) to move at least one shock wave emitter 1806 closer to a lesion within a body lumen, such as a valve, when expanded. Catheter 1800 is configured such that the at least one shock wave emitter 1806 can positioned closely to a lesion within a valve (such as the valve depicted in FIGS. 3A and 3B) without relying on typical positioning methods such as steering or centering with occlusion. Catheter 1800 may also be configured such that blood flow is not fully occluded when the plurality of expandable members, including expandable member 1808 and expandable member 1810, are expanded. Catheter 1000 thus enables more effective treatment of lesions within the body by allowing users to impact lesions with relatively more powerful shock waves.

Catheter 1800 includes a catheter body 1812 and an elongate member 1802. An enclosure 1804 is mounted to the elongate member 1802. Enclosure 1804 is optionally also mounted to the catheter body 1812. In some examples, the elongate member 1802 forms part of the catheter body 1812. The enclosure 1804 may be compliant such that it stretches when pressurized, non-compliant such that it does not stretch or stretches minimally when pressurized with a fluid, or semi-compliant. At least one shock wave emitter 1806 may be carried by the elongate member 1802 and disposed within the enclosure 1804. In some examples, the elongate member 1802 may be a distal end portion of the catheter body. In some examples, the elongate member 1802 may extend within a lumen along at least a portion of the length of catheter body 1812. The expandable members 1808 and 1810 may be configured to expand to contact the enclosure 1804 and push the enclosure 1804, elongate member 1802, and the at least one shock wave emitter 1806 laterally toward a lesion. A distal end 1818 of expandable member 1808 and a distal end 1820 of expandable member 1810 may be connected to a distal end 1814 of the enclosure 1804. Enclosure 1804, expandable member 1810, and expandable member 1808 may thus form a connected fillable/inflatable space into which a conductive fluid can be introduced to expand the expandable members 1808 and 1810 and also facilitate shock wave generation. Connecting the distal ends of the expandable members 1808 and 1810 to the distal end of enclosure 1804 may enable easier navigation of catheter 1800 within the vasculature. For instance, connecting expandable members 1808 and 1810 to the enclosure 1804 may prevent the expandable members 1808 and 1810 from getting caught on aspects of the vasculature and/or being bent proximally away from the enclosure 1804.

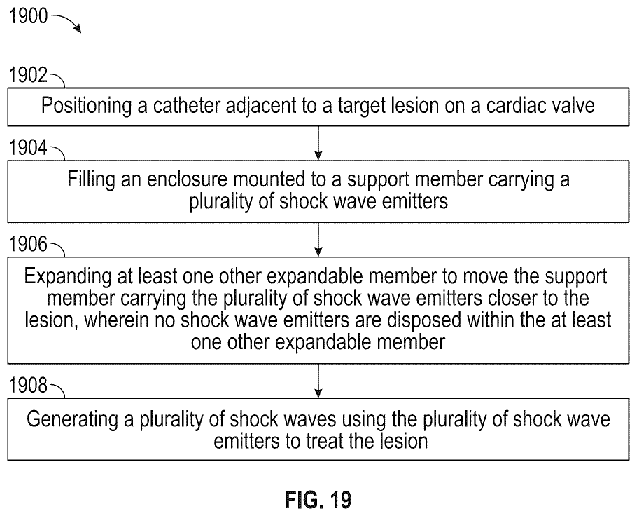

As discussed throughout catheters having expandable members for moving shock wave emitters relatively closer to lesions within the body may be configured such that they are particularly useful for treatment of lesions within cardiac valves. Different cardiac valves have different morphological characteristics. For instance, some valves may have three leaflets and three corresponding cusps. Some valves may have two leaflets and two corresponding cusps. Some valves may have three leaflets but only two cusps. During use, the expandable members on the catheters disclosed herein may be selectively expanded to move shock wave emitters closer to lesions based on the morphology of the valve being treated. FIG. 19 illustrates aspects of a method 1900 for selectively expanding one or more expandable members of the catheters disclosed herein to treat lesions within a valve. At block 1902, method 1900 may include positioning a catheter adjacent to a target lesion on a cardiac valve. At block 1904, method 1900 may include filling an enclosure mounted to a support member carrying a plurality of shock wave emitters. At block 1906, method 1900 may include expanding at least one expandable member to move the support member carrying the plurality of shock wave emitters closer to the lesion. In some examples, no shock wave emitters are disposed within the at least one expandable member. In some examples, the cardiac valve may be a mitral valve, which only has two cusps, and in such examples only one of the at least one expandable member is expanded. In some examples, the cardiac valve is an aortic valve, which has three cusps, and in such examples the at least one expandable member includes at least two expandable members that are both expanded. In any of these examples, the at least one expandable member is configured to expand to contact the enclosure mounted to the support member to push the support member and shock wave emitter(s) closer to a lesion.

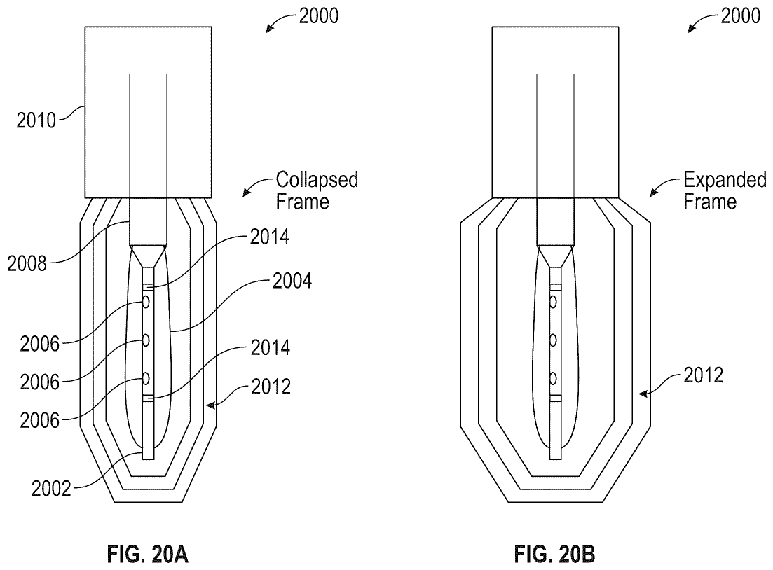

FIGS. 20A and 20B illustrates aspects of an exemplary catheter 2000 that may be used for catheter 10 of FIG. 1. Catheter 2000 may be configured to enable a user to treat lesions within or proximate to a valve (e.g., a cardiac valve) without blocking blood flow through the valve. Catheter 2000 may be configured to enable a user to expand or open a valve, for instance, by pushing the leaflets of the valve outward, and to generate one or more shock waves from radially within the valve directed outward toward the leaflets and/or annulus of the valve. Catheter 2000 may include a catheter body 2008 and an elongate member 2002 extending from a distal portion of the elongate member. A plurality of shock wave emitters 2006 may be positioned on the elongate member 2002 within an enclosure 2004 sealed to the elongate member. Catheter 2000 may include an expandable frame 2012 at least partially circumscribing the enclosure 2004. The expandable frame may be configured to expand from a collapsed position shown in FIG. 20A to an expanded position shown in FIG. 20B. In the expanded position, the expandable frame may be positioned further from the enclosure 2004 than in the collapsed position. The expandable frame 2012 may be a wire frame or a mesh. The expandable frame 2012 may be configured to self-expand when translated distally of a distal end of an outer sheath 2010. The expandable frame may be formed from a shape-memory material, such as nitinol. In some examples, the expandable frame is configured to collapse when translated proximally into the distal end of the outer sheath 2010. In some examples, catheter 2000 includes one or more marker bands 2014 configured to enable a user to determine a position of the one or more shock wave emitters within the body (e.g., relative to a lesion). For instance, a marker band 2014 may be positioned proximally of a plurality of shock wave emitters 2006 and another marker band 2014 may be positioned distally of the plurality of shock wave emitters 2006. The catheter may be positioned such that the marker band 2014 positioned distally of the plurality of shock wave emitters 2006 is positioned distally of a valve annulus and the marker band 2014 positioned proximally of the plurality of shock wave emitters 2006 is positioned proximally of the valve annulus. Thus, a user may determine that the plurality of shock wave emitters are aligned with the valve annulus.

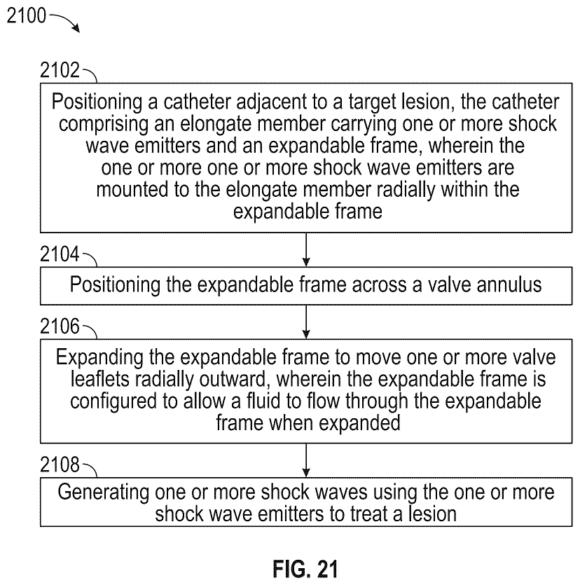

FIG. 21 illustrates aspects of an exemplary method 2100 of generating shock waves for treating lesions within a valve. At block 2102, method 2100 may include positioning a catheter adjacent to a target lesion. The target lesion may be a calcification or other lesion on a valve (e.g., aortic or mitral valve). The catheter may include an elongate member carrying one or more shock wave emitters and an expandable frame. The one or more one or more shock wave emitters may be mounted to the elongate member radially within the expandable frame. At block 2104, method 2100 may include positioning the expandable frame across a valve annulus such that the plurality of shock wave emitters are aligned with the valve annulus. At block 2106, method 2100 may include expanding the expandable frame to move one or more valve leaflets radially outward. The expandable frame may be configured to allow a fluid to flow through the expandable frame when expanded. For instance, the expandable frame may be a wire frame or mesh frame configured to enable fluid to pass by or through the frame. At block 2108, method 2100 may include generating one or more shock waves using the one or more shock wave emitters to treat a lesion.

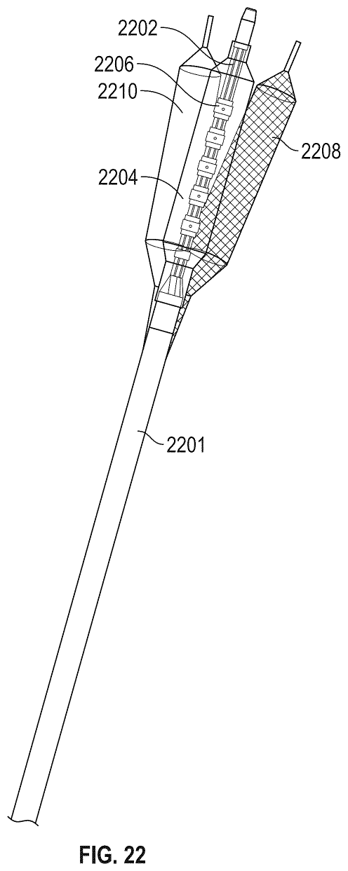

FIG. 22 illustrates aspects of a catheter 2200 that may be used for catheter 10 of FIG. 1. Catheter 1800 includes a plurality of expandable members including expandable member 2208 and expandable member 2210, that enable a user (e.g., a surgeon or other medical professional) to move at least one shock wave emitter 2206 closer to a lesion within a body lumen, such as a valve, when expanded. Expandable member 2208 may be an expandable frame, such as a wire frame or a mesh, that allows fluid to flow by or through the frame when the frame is expanded. Expandable member 2210 may be an expandable balloon or other enclosure configured to be filled/inflated with a fluid. Using at least one expandable frame as one of the expandable members may allow for blood flow to continue when the expandable members are expanded during treatment. Catheter 2200 thus enables more effective treatment of lesions within the body by allowing users to impact lesions with relatively more powerful shock waves.

Catheter 2200 includes a catheter body 2201 and an elongate member 2202. An enclosure 2204 is mounted to the elongate member 2202. Enclosure 2204 is optionally also mounted to the catheter body 2201. In some examples, the elongate member 2202 forms part of the catheter body 2201. The enclosure 2204 may be compliant such that it stretches when pressurized, non-compliant such that it does not stretch or stretches minimally when pressurized with a fluid, or semi-compliant. At least one shock wave emitter 2206 may be carried by the elongate member 2202 and disposed within the enclosure 2204. In some examples, the elongate member 2202 may be a distal end portion of the catheter body. In some examples, the elongate member 2202 may extend within a lumen along at least a portion of the length of catheter body 2201. The expandable members 2208 and 2210 may be configured to expand to contact the enclosure 2204 and push the enclosure 2204, elongate member 2202, and the at least one shock wave emitter 2206 laterally toward a lesion.

Although the electrode assemblies and catheter devices described herein have been discussed primarily in the context of treating coronary occlusions, such as lesions in vasculature, the electrode assemblies and catheters herein can be used for a variety of occlusions, such as occlusions in the peripheral vasculature (e.g., above-the-knee, below-the-knee, iliac, carotid, etc.). For further examples, various embodiments may be used for treating soft tissues, such as cancer and tumors (i.e., non-thermal ablation methods), blood clots, fibroids, cysts, organs, scar and fibrotic tissue removal, or other tissue destruction and removal. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the electrode assemblies and catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It should be noted that the elements and features of the example catheters illustrated throughout this specification and drawings may be rearranged, recombined, and modified without departing from the present invention. For instance, while this specification and drawings describe and illustrate catheters having several example balloon designs, the present disclosure is intended to include catheters having a variety of balloon configurations. The number, placement, and spacing of the electrode pairs of the shock wave generators can be modified without departing from the subject invention. Further, the number, placement, and spacing of balloons of catheters can be modified without departing from the subject invention.

It should be understood that the foregoing is only illustrative of the principles of the invention, and that various modifications, alterations and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

### CLAUSES

1. A catheter for generating shock waves comprising:
   a first elongate member;
   an enclosure mounted to the first elongate member;
   at least one shock wave emitter carried by the first elongate member and disposed within the enclosure;
   a first set of radiopaque markers positioned on the first elongate member within the first enclosure;
   a second elongate member;
   an expandable member mounted to the second elongate member and configured to expand to contact the enclosure mounted to the first elongate member; and
   a second set of radiopaque markers positioned on the second elongate member within the expandable member, wherein the second set of radiopaque marks has a different arrangement than the first set of radiopaque markers.
2. The catheter of clause 1, wherein the first elongate member extends distally from a catheter body.
3. The catheter of any one of clauses 1-2, wherein the first elongate member is a distal portion of a catheter body.
4. The catheter of any one of clauses 1-3, wherein the different arrangement of radiopaque markers comprises a different number of radiopaque markers.
5. The catheter of clause 4, wherein the different arrangement of radiopaque markers comprises a different spacing between the radiopaque markers.
6. The catheter of any one of clauses 4-5, wherein the first set of radiopaque markers and the second set of radiopaque markers are arranged on the first and second elongate members such that:
   the first set of radiopaque markers appears colinear with the second set of radiopaque markers when viewed from a first perspective using radiographic imaging.
7. The catheter of clause 6, wherein the first set of radiopaque markers appears non-colinear with the second set of radiopaque markers when viewed from a second perspective using radiographic imaging.
8. The catheter of any one of clauses 1-7, wherein the enclosure is non-compliant.
9. The catheter of any one of clauses 1-8, wherein the expandable member mounted to the second elongate member is an enclosure configured to expand when inflated with a fluid.
10. The catheter of clause 9, wherein the catheter comprises a first fluid conduit for filling the enclosure mounted to the first elongate member and a second fluid conduit for filling the enclosure mounted to the second elongate member so that the enclosure mounted to the first elongate member can be filled independently of the enclosure mounted to the second elongate member.
11. The catheter of any one of clauses 1-10, wherein the first elongate member comprises a guidewire lumen.
12. The catheter of any one of clauses 1-11, wherein the second elongate member comprises a guidewire lumen.
13. The catheter of any one of clauses 1-12, wherein a distal end of the first elongate member is free of a distal end of the second elongate member.
14. The catheter of any one of clauses 1-13, wherein the first elongate member and the second elongate member extend proximally within the same lumen along at least a portion of the length of the catheter body.
15. The catheter of any one of clauses 1-14, wherein the at least one shock wave emitter is located on an opposite side of the first elongate member from the second elongate member.
16. A method for positioning a shock wave generating catheter closer to a target lesion, the method comprising:
   advancing a catheter within a lumen to the target lesion;
   orienting the catheter within the lumen such that a first elongate member comprising a first set of radiopaque markers is positioned relatively closer to the target lesion than a second elongate member comprising a second set of radiopaque markers;
   moving a first elongate member of the catheter closer to the target lesion by expanding an expandable member such that the expandable member pushes the first elongate member closer to the target lesion; and
   generating one or more shock waves using at least one shock wave emitter of the catheter positioned on the first elongate member.
17. The method of clause 16, comprising rotating the catheter to position the first elongate member of the catheter adjacent to the target lesion.
18. The method of any one of clauses 16-17, wherein the expandable member comprises an enclosure, and wherein expanding the expandable member connected to the second elongate member comprises inflating the enclosure mounted to a distal end of the second elongate member to contact an enclosure mounted to the first elongate member.
19. The method of any one of clauses 16-17, wherein the expandable member comprises an expandable frame, and wherein expanding the expandable member comprises moving a movable shaft connected to the expandable frame in a proximal direction.
20. The method of any one of clauses 16-19, wherein the at least one shock wave emitter of the catheter is positioned within an enclosure connected to the first elongate member.
21. A system for generating shock waves, the system comprising:
   a shock wave energy generator; and
   the catheter of any one of clauses 1-20.
22. A catheter for generating shock waves comprising:
   a catheter body;
   a tube-shaped enclosure sealed to a distal end of the catheter body, the tube-shaped enclosure comprising an outer cylindrical wall, an inner cylindrical wall, and a fillable region between the outer cylindrical wall and the inner cylindrical wall configured to be filled with a fluid, wherein a distal portion of the catheter body is positioned within the fillable region; and
   at least one shock wave emitter disposed on the catheter body within the fillable region between the outer cylindrical wall and the inner cylindrical wall.
23. The catheter of clause 22, wherein the inner cylindrical wall defines an open channel when the tube-shaped enclosure is filled with a fluid.
24. The catheter of clause 23, wherein the open channel is configured to allow body fluid flowing within a body lumen to flow through the open channel when the catheter is disposed in the lumen and the tube-shaped enclosure is filled with a fluid.
25. The catheter of any one of clauses 22-24, wherein the tube-shaped enclosure is semi-compliant.
26. The catheter of clause any one of clauses 22-25, wherein the tube-shaped enclosure is non-compliant balloon.
27. The catheter any one of clauses 22-26, wherein a compliance of the outer cylindrical wall is different than a compliance of the inner cylindrical wall.
28. The catheter any one of clauses 22-27, wherein a distal portion of the tube-shaped enclosure comprises a tapered region.
29. The catheter of any one of clauses 22-28, wherein the outer cylindrical wall has a diameter of between 10 millimeters and 30 millimeters.
30. The catheter of any one of clauses 22-29, wherein the inner cylindrical wall has a diameter of at least 6 millimeters.
31. The catheter of any one of clauses 22-30, wherein the tube-shaped enclosure comprises a proximally extending cylindrical wall at a proximal end of the balloon and a distally extending cylindrical wall at a distal end of the balloon, wherein the proximally extending cylindrical wall and the distally extending cylindrical wall are sealed to the catheter body.
32. The catheter of any one of clauses 22-231, wherein the tube-shaped enclosure comprises a shape memory material.
33. The catheter of any one of clauses 22-32, the catheter comprises a braided outer shaft on a proximal portion of the catheter body.
34. The catheter of any one of clauses 22-33, wherein the at least one shock wave emitter is configured to emit shock waves toward the outer cylindrical wall.
35. The catheter of clause 34, wherein the at least one shock wave emitter is positioned at circumferential location of the catheter body that is closest to the outer cylindrical wall.
36. A method for positioning a shock wave generating catheter closer to a target treatment area, the method comprising:
   advancing a catheter comprising a tube-shaped enclosure mounted to a catheter body within a lumen;
   positioning a distal portion of the catheter such that at least one shock wave emitter of the catheter is positioned adjacent to a target treatment area;
   filling a tube-shaped enclosure to form an open channel through which body fluid flows;
   generating one or more shock waves using at least one shock wave emitter of the catheter.
37. The method of clause 36, comprising:
   deflating the tube-shaped enclosure;
   rotating the catheter to position the at least one shock wave emitter adjacent to a different target treatment area;
   filling the tube-shaped enclosure; and
   generating one or more additional shock waves using the at least one shock wave emitter of the catheter.
38. The method of any one of clauses 36-37, wherein positioning a distal portion of the catheter such that at least one shock wave emitter of the catheter is positioned adjacent to a target treatment area comprises:
   positioning the distal portion of the catheter across a valve annulus.
39. The method of any one of clauses 36-38, wherein positioning a distal portion of the catheter such that at least one shock wave emitter of the catheter is positioned adjacent to a target treatment area comprises:
   positioning the distal portion of the catheter adjacent to an aortic valve leaflet or a mitral valve leaflet.
40. A system for generating shock waves, the system comprising:
   a shock wave energy generator; and
   the catheter of any one of clauses 22-39.
41. A catheter for generating shock waves comprising:
   an elongate member;
   an enclosure mounted to the elongate member;
   at least one shock wave emitter carried by the elongate member and disposed within the enclosure;
   a first expandable member extending away from the elongate member in a first direction and configured to expand to contact the enclosure mounted to the elongate member; and
   a second expandable member extending away from the elongate member and the first expandable member in a second direction and configured to expand to contact the enclosure mounted to the elongate member.
42. The catheter of clause 41, wherein the first expandable member is mounted to a second elongate member and the second expandable member is mounted to a third elongate member.
43. The catheter of any one of clauses 41-42, wherein the first expandable member and the second expandable member have a substantially equal outer diameter when filled with a fluid.
44. The catheter of clause 43, wherein the enclosure has substantially the same outer diameter as the first expandable member and the second expandable member when filled with a fluid.
45. The catheter of any one of clauses 41-44, wherein at least one radiopaque marker is positioned on the elongate member.
46. A system for generating shock waves, the system comprising:
   a shock wave energy generator; and
   the catheter of any one of clauses 41-45.
47. A catheter for generating shock waves comprising:
   a first elongate member;
   an enclosure mounted to the first elongate member;
   at least one shock wave emitter carried by the first elongate member and disposed within the enclosure;
   a second elongate member; and
   an expandable frame mounted to the second elongate member and configured to expand to contact the enclosure mounted to the first elongate member, wherein the expandable frame is configured such that, when the catheter is located in a blood vessel and the expandable frame is expanded, blood can flow through the expandable frame.
48. The catheter of clause 47, wherein the expandable frame comprises a plurality of expandable members.
49. The catheter of any one of clauses 47-49, wherein the expandable frame comprises a coiled member.
50. The catheter of any one of clauses 47-51, comprising a moveable shaft connected to the expandable frame configured to expand and collapse the expandable frame.
51. A catheter for generating shock waves comprising:
   an inner elongate member;
   an outer elongate member extending radially outwardly of the inner elongate member, wherein the outer elongate member is translatable relative to the inner elongate member between a retracted position and an extended position;
   at least one support member positioned at least partially between the inner elongate member and the outer elongate member when the outer elongate member is in the extended position, wherein the at least one support member is configured to move outwardly from the inner elongate member when the outer elongate member is translated from the extended position to the retracted position; and
   at least one shock wave emitter mounted to the at least one expandable emitter support member.
52. The catheter of clause 53, wherein at least one support member comprises a pre-shaped memory material configured to move outwardly from the inner elongate member when the outer elongate member is translated from the extended position to the retracted position.
53. The catheter of clause 54, wherein the at least one support member comprises a lumen, wherein the pre-shaped memory material is positioned within the lumen.
54. The catheter of any one of clauses 53-55, comprising a user-engageable locking member configured to lock the outer elongate member to the inner elongate member such that the outer elongate member is not translatable relative to the inner elongate member.
55. The catheter of clause 56, wherein rotating the locking member in a first direction locks the outer elongate member to the inner elongate member.
56. The catheter of any one of clauses 53-58, wherein the at least one support member is positioned within at least one groove of the inner elongate member.
57. The catheter of any one of clauses 53-59, comprising an enclosure mounted to the at least one expandable member, wherein the at least one shock wave emitter is positioned within the enclosure.
58. A method for treating lesions within the body, the method comprising:
   advancing a catheter within a body lumen to a target lesion;
   sliding an elongate member proximally to deploy one or more elongate support members such that the one or more elongate support members move outwardly from a longitudinal axis of the catheter;
   introducing a conductive fluid into one or more enclosures provided on the one or more elongate support members;
   generating one or more shock waves using at least one shock wave emitter positioned on at least one of the one or more elongate support members.
59. The method of clause 61, comprising: rotating a locking member to unlock the outer elongate member from a second elongate member prior to sliding the outer elongate member proximally.
60. The method of any one of clauses clause 61-62, comprising: manipulating the catheter to position a portion of each of the one or more elongate support members in a respective valve cusp.
61. The method of any one of clauses clause 61-63, comprising: sliding the elongate member distally to collapse the elongate support members inwardly.
62. The method of any one of clauses clause 61-64, wherein the fluid is introduced into each of the one or more enclosures independently of one or more of the other enclosures.
63. The method of any one of clauses clause 61-65, wherein the fluid is introduced into each of the one or more enclosures simultaneously.

1. A catheter for generating shock waves comprising:
a first elongate member;
an enclosure mounted to the first elongate member;
at least one shock wave emitter carried by the first elongate member and disposed within the enclosure;
a first set of radiopaque markers positioned on the first elongate member within the first enclosure;
a second elongate member;
an expandable member mounted to the second elongate member and configured to expand to contact the enclosure mounted to the first elongate member; and
a second set of radiopaque markers positioned on the second elongate member within the expandable member, wherein the second set of radiopaque marks has a different arrangement than the first set of radiopaque markers.

2. The catheter of claim 1, wherein the different arrangement of radiopaque markers comprises a different number of radiopaque markers.

3. The catheter of claim 2, wherein the different arrangement of radiopaque markers comprises a different spacing between the radiopaque markers.

4. The catheter of claim 2, wherein the first set of radiopaque markers and the second set of radiopaque markers are arranged on the first and second elongate members such that:
the first set of radiopaque markers appears colinear with the second set of radiopaque markers when viewed from a first perspective using radiographic imaging.

5. The catheter of claim 4, wherein the first set of radiopaque markers appears non-colinear with the second set of radiopaque markers when viewed from a second perspective using radiographic imaging.

6. The catheter of claim 1, wherein the expandable member mounted to the second elongate member is an enclosure configured to expand when inflated with a fluid.

7. The catheter of claim 6, wherein the catheter comprises a first fluid conduit for filling the enclosure mounted to the first elongate member and a second fluid conduit for filling the enclosure mounted to the second elongate member so that the enclosure mounted to the first elongate member can be filled independently of the enclosure mounted to the second elongate member.

8. The catheter of claim 1, wherein either or both of the first elongate member and the second elongate member comprises a guidewire lumen.

9. The catheter of claim 1, wherein a distal end of the first elongate member is free of a distal end of the second elongate member.

10. The catheter of claim 1, wherein the first elongate member and the second elongate member extend proximally within the same lumen along at least a portion of the length of the catheter body.

11. The catheter of claim 1, wherein the at least one shock wave emitter is located on an opposite side of the first elongate member from the second elongate member.

12. The catheter of claim 1, wherein the expandable member mounted to the second elongate member is an expandable frame configured to allow a fluid to pass through the frame when the frame is expanded.

13. The catheter of claim 12, comprising a moveable shaft configured such that translation of the moveable shaft in a first direction causes the expandable frame to expand and translation of the moveable shaft in a second direction causes the expandable frame to collapse.

14. The catheter of claim 12, wherein the expandable frame comprises at least one of a wire frame wire and a mesh.

15. The catheter of claim 12, wherein the expandable frame is configured to be self-expanding.

16. The catheter of claim 1, wherein a distal end of the expandable member is positioned proximally of a distal end of the enclosure.

17. A method for positioning a shock wave generating catheter closer to a target lesion, the method comprising:
advancing a catheter within a lumen to the target lesion;
orienting the catheter within the lumen such that a first elongate member comprising a first set of radiopaque markers is positioned relatively closer to the target lesion than a second elongate member comprising a second set of radiopaque markers;
moving a first elongate member of the catheter closer to the target lesion by expanding an expandable member such that the expandable member pushes the first elongate member closer to the target lesion; and
generating one or more shock waves using at least one shock wave emitter of the catheter positioned on the first elongate member.

18. The method of claim 17, comprising rotating the catheter to position the first elongate member of the catheter adjacent to the target lesion.

19. The method of claim 17, wherein the expandable member comprises an enclosure, and wherein expanding the expandable member connected to the second elongate member comprises inflating the enclosure mounted to a distal end of the second elongate member to contact an enclosure mounted to the first elongate member.

20. The method of claim 17, wherein the expandable member comprises an expandable frame, and wherein expanding the expandable member comprises moving a movable shaft connected to the expandable frame in a proximal direction.

21. The method of claim 17, wherein the at least one shock wave emitter of the catheter is positioned within an enclosure connected to the first elongate member.
